(19) United States
(12) Patent Application Publication
(10) Pub. No.: US 2017/0119349 A1
(43) Pub. Date: May 4, 2017

(54) PIEZOELECTRIC ELEMENT, PIEZOELECTRIC MODULE, ELECTRONIC APPARATUS, AND PIEZOELECTRIC ELEMENT MANUFACTURING METHOD

(71) Applicant: SEIKO EPSON CORPORATION, Tokyo (JP)

(72) Inventors: Hiromu MIYAZAWA, Azumino-shi (JP); Hiroshi ITO, Suwa-shi (JP); Tomoaki NAKAMURA, Chino-shi (JP); Masayoshi YAMADA, Chino-shi (JP); Sayaka YAMASAKI, Suwa-shi (JP); Tsukasa FUNASAKA, Shiojiri-shi (JP)

(73) Assignee: SEIKO EPSON CORPORATION, Tokyo (JP)

(21) Appl. No.: 15/332,381

(22) Filed: Oct. 24, 2016

(30) Foreign Application Priority Data
Oct. 29, 2015 (JP) 2015-213454

(51) Int. Cl. A61B 8/00; H01L 41/08; H01L 41/113; G01S 7/52; H01L 41/29; H01L 41/27; B06B 1/06; G01S 15/89; H01L 41/047; H01L 41/187

(52) U.S. Cl. CPC A61B 8/4494; H01L 41/047; H01L 41/081; H01L 41/1132; H01L 41/1876; H01L 41/29; H01L 41/27; B06B 1/0622; A61B 8/54; G01S 15/8925; G01S 7/52082

(57) ABSTRACT

A receiving transducer includes: a flexible portion; a piezoelectric film provided on the flexible portion; a first electrode provided between a first surface of the flexible portion, on which the piezoelectric body is provided, and a second surface of the piezoelectric film that is a surface not facing the flexible portion; and a second electrode that is provided between the first and second surfaces and that faces the first electrode with a gap interposed therebetween in plan view as viewed from the thickness direction of the flexible portion.

### BACKGROUND

1. Technical Field

The present invention relates to a piezoelectric element, a piezoelectric module, an electronic apparatus, a piezoelectric element manufacturing method, and the like.

2. Related Art

A piezoelectric element has been known in which a piezoelectric body is formed on a flexible film and the flexible film is vibrated by a driving voltage applied to the piezoelectric body (for example, refer to JP-A-2002-271897).

JP-A-2002-271897 discloses an ultrasonic transducer (piezoelectric element) in which a piezoelectric layer is formed on a flexible film and first and second electrodes are disposed on the same surface of the piezoelectric layer so as to face each other.

In the ultrasonic transducer disclosed in JP-A-2002-271897, the first and second electrodes are provided on the surface of the piezoelectric layer. The ultrasonic transducer having such a structure is formed by forming a piezoelectric body on the flexible film and providing electrodes on the piezoelectric body. However, since the piezoelectric body is deteriorated when forming electrodes on the piezoelectric body, there is a problem that the piezoelectric characteristics of the piezoelectric body are degraded (for example, a value of a piezoelectric e constant is reduced).

### SUMMARY

An advantage of some aspects of the invention is to provide a piezoelectric element including a piezoelectric body having enhanced piezoelectric characteristics, a piezoelectric module, an electronic apparatus, and a piezoelectric element manufacturing method.

A piezoelectric element according to one application example of the invention includes: a flexible film; a piezoelectric body provided on the flexible film; a first electrode provided between a first surface of the flexible film, on which the piezoelectric body is provided, and a second surface of the piezoelectric body not facing the flexible film; and a second electrode that is provided between the first and second surfaces and that faces the first electrode with a first gap interposed therebetween in plan view as viewed from a thickness direction of the flexible film.

In this application example, for the piezoelectric body provided on the flexible film, the first and second electrodes are disposed so as to face each other with the first gap interposed therebetween in plan view as viewed from the thickness direction of the flexible film. That is, the piezoelectric body is provided in the first gap between the first and second electrodes.

In such a configuration, it is possible to suppress the degradation of the piezoelectric characteristics of the piezoelectric body, compared with a configuration in which the first and second electrodes are provided on the piezoelectric body. That is, in a case where the first and second electrodes are provided after providing the piezoelectric body, the piezoelectric body is deteriorated when the first and second electrodes are formed on the piezoelectric body. Accordingly, the value of the piezoelectric e constant is reduced. In contrast, for example, in a case where the first and second electrodes are provided on the flexible film and the piezoelectric body is provided thereon, deterioration of the piezoelectric body due to electrode formation can be prevented since the first and second electrodes are formed before forming the piezoelectric body. In addition, a lower layer of the piezoelectric body may be formed on the flexible film, and then the first and second electrodes may be formed and the piezoelectric body may be formed thereon. In this case, deterioration due to formation of the first and second electrodes occurs in the lower layer of the piezoelectric body, but there is no deterioration in the upper layer of the piezoelectric body. Accordingly, since the deterioration of the piezoelectric body can be suppressed compared with a case where the first and second electrodes are formed on the second surface (surface) of the piezoelectric body, it is possible to enhance the piezoelectric characteristics of the piezoelectric body.

In addition, in this application example, since the piezoelectric body is interposed between the first and second electrodes, it is possible to suppress dielectric breakdown when applying a voltage between the first and second electrodes (in particular, in the case of performing polarization processing by applying a high voltage between the first and second electrodes).

In the piezoelectric element according to the application example, it is preferable that the first and second electrodes are provided between the flexible film and the piezoelectric body.

In the application example with this configuration, the first and second electrodes are provided between the flexible film and the piezoelectric body. In such a configuration, it is possible to form the piezoelectric body after forming the first and second electrodes on the flexible film. That is, since neither the first electrode nor the second electrode is formed on the piezoelectric body, deterioration of the piezoelectric body due to formation of the first electrodes and second electrodes is suppressed. Accordingly, it is possible to enhance the piezoelectric characteristics.

In the piezoelectric element according to the application example, it is preferable that the first and second electrodes are embedded in the piezoelectric body.

In the application example with this configuration, the first and second electrodes are embedded in the piezoelectric body. In such a configuration, it is possible to form the first and second electrodes after forming a part of the piezoelectric body on the flexible film and then form a remaining portion of the piezoelectric body. In this case, in a part of the piezoelectric body formed on the flexible film, the piezoelectric characteristics are degraded since the first and second electrodes are formed on the part of the piezoelectric body. However, in the remaining portion of the piezoelectric body formed on the first and second electrodes, degradation of the piezoelectric characteristics is suppressed. Therefore, for example, compared with a case where the piezoelectric body is formed on the flexible film and the first and second electrodes are formed on the surface of the piezoelectric body, it is possible to enhance the piezoelectric characteristics of the piezoelectric body.

In the piezoelectric element according to the application example, it is preferable that the first and second electrodes are provided within a plane parallel to the first surface.

In the application example with this configuration, the first and second electrodes are provided within a plane parallel to the first surface. In this case, since it is possible to form the first and second electrodes simultaneously, it is possible to simplify the process of manufacturing the piezoelectric element.

The first electrode or the second electrode is formed, for example, by coating an electrode material on the surface of a part of the flexible film or the piezoelectric body using a sputtering method or a vacuum deposition method and then performing patterning to form an electrode shape. Accordingly, in the case of forming the first and second electrodes at different height positions (case where the first and second electrodes are not provided on the same plane), for example, in a case where a part of the piezoelectric body is formed on the flexible film, the first electrode is then formed, another part of the piezoelectric body is formed on the upper surface of the first electrode, the second electrode is formed on the upper surface of another part of the piezoelectric body, and then a remaining portion of the piezoelectric body is formed, deterioration of the piezoelectric body further proceeds since two electrode layer forming steps are included. In contrast, in a case where the first and second electrodes are provided within the same plane as described above, it is possible to form the first and second electrodes simultaneously as described above. Therefore, it is possible to suppress the deterioration of the piezoelectric body.

In the piezoelectric element according to the application example, it is preferable that the first electrode has a first end surface facing the second electrode, the second electrode has a second end surface facing the first electrode, and the first and second end surfaces are parallel to each other.

In the case of holding electric charges in the first and second electrodes facing each other, the electric charges are held around positions, at which the distance between electrodes is the shortest, in regions of the first and second electrodes facing each other. Accordingly, in the application example with the configuration described above, displacement current flows between the first end surface of the first electrode and the second end surface of the second electrode that are disposed in parallel to each other. For example, in the case of acquiring (detecting) the displacement current output from the piezoelectric body due to the displacement of the flexible film in the form of a voltage, if the first and second electrodes are disposed in parallel to each other, it is possible to detect the displacement current in a wide range of the piezoelectric body. Therefore, it is possible to improve the voltage detection accuracy. In addition, for example, in the case of driving the piezoelectric body by applying a driving voltage between the first and second electrodes, it is possible to distort the piezoelectric body uniformly since the displacement current flows uniformly in the wide range of the piezoelectric body.

In the piezoelectric element according to the application example, it is preferable to further include at least one or more intermediate electrodes that are provided between the first and second electrodes in plan view and that face each of the first and second electrodes with a second gap interposed therebetween in plan view.

In the application example with this configuration, one or more intermediate electrodes are provided between the first and second electrodes in plan view. Accordingly, the electrostatic capacitance is formed not only between the first electrode and the intermediate electrode and between the second electrode and the intermediate electrode but also between the intermediate electrodes in a case where a plurality of intermediate electrodes are further provided. In such a configuration, since the areas of the facing regions of electrodes facing each other are increased, it is possible to increase the total electrostatic capacitance of the piezoelectric element. Therefore, since it is possible to suppress the influence of the stray capacitance of an external circuit, it is possible to avoid a voltage drop in the received signal.

In the piezoelectric element according to the application example, it is preferable that the piezoelectric body is formed of a perovskite type transition metal oxide.

In the application example with this configuration, a perovskite type transition metal oxide is used as a material of the piezoelectric body. The perovskite type transition metal oxide is a piezoelectric material having enhanced piezoelectric characteristics (high piezoelectric e constant). Therefore, it is possible to increase the voltage output from the piezoelectric body when the flexible film is displaced.

In the piezoelectric element according to the application example, it is preferable that the piezoelectric body contains Pb, Zr, and Ti.

In the application example with this configuration, the piezoelectric body contains Pb, Zr, and Ti. As such a piezoelectric body, for example, lead zirconate titanate (PZT) can be mentioned. Among perovskite type transition metal oxides, the lead zirconate titanate (PZT) has particularly enhanced piezoelectric characteristics. Therefore, it is possible to further increase the voltage output from the piezoelectric body when the flexible film is displaced.

In the piezoelectric element according to the application example, it is preferable that the flexible film includes a first layer in contact with the piezoelectric body and the first layer is formed of a transition metal oxide.

Here, the first layer maybe one layer of a flexible film configured to include a plurality of layers, and the flexible film may be formed as one layer (only a first layer of the transition metal oxide).

In the application example with the configuration described above, the first layer of the flexible film in contact with the piezoelectric body is formed of a transition metal oxide. In the case of forming a piezoelectric body on such a flexible film, it is possible to suppress the diffusion of an element having high vapor pressure, such as Pb, contained in the piezoelectric body. In addition, since it is easy to form the piezoelectric body having (100) orientation, it is possible to enhance the piezoelectric characteristics of the piezoelectric body.

In the piezoelectric element according to the application example, it is preferable that the first layer is formed of ZrO₂.

In the application example with this configuration, since the first layer is formed of ZrO₂, it is possible to suppress the diffusion of an element having high vapor pressure, such as Pb, contained in the piezoelectric body. In addition, since it becomes easy to make the crystal orientation of the piezoelectric body be the (100) orientation, it is possible to further enhance the piezoelectric characteristics of the piezoelectric body.

More specifically, if Ti having a thickness of 10 nm or less or BiFeTiO₃ having a thickness of 100 nm or less is laminated on ZrO₂ and then the piezoelectric body is formed on the Ti or BiFeTiO₃, the piezoelectric body is (100) preferentially oriented.

In addition, since the Ti or BiFeTiO₃ becomes an oxide film after being subjected to heat processing in the manufacturing process, it is requested to have a high insulation property. That is, if a conductive region is present between the first and second electrodes, it is not possible to obtain high reception sensitivity.

In the piezoelectric element according to the application example, it is preferable that the first gap is 2 μm or more and 8 μm or less.

In the application example with this configuration, the gap between the first and second electrodes is 2 μm or more and 8 μm or less. In a case where the first gap between the first and second electrodes is less than 2 μm, the voltage output from the piezoelectric body with respect to the amount of distortion of the piezoelectric body is reduced. In this case, for example, in the case of detecting the amount of displacement of the flexible film based on the voltage output from the piezoelectric body, the detection accuracy is reduced since the output voltage is reduced. On the other hand, in a case where the first gap between the first and second electrodes is larger than 8 μm, it is necessary to set a high voltage as an application voltage when performing polarization processing on the piezoelectric body. In contrast, in the application example with the configuration described above, since the first gap of the range described above is provided, it is possible to increase the voltage output from the piezoelectric body with respect to the amount of distortion of the piezoelectric body. In addition, it is possible to keep the application voltage at the time of polarization processing in a practical range.

A piezoelectric module according to one application example of the invention includes: a flexible film; a piezoelectric body having a first surface in contact with the flexible film and a second surface opposite to the first surface; a first electrode provided between the first and second surfaces of the piezoelectric body; a second electrode that is provided between the first and second surfaces of the piezoelectric body and that faces the first electrode with a first gap interposed therebetween in plan view as viewed from a thickness direction of the flexible film; and a wiring substrate having a terminal unit to which the first and second electrodes are electrically connected.

The piezoelectric module according to the application example includes the piezoelectric element described above and the wiring substrate having a terminal unit to which the first and second electrodes of the piezoelectric element are electrically connected. Therefore, as in the application examples described above, it is possible to enhance the piezoelectric characteristics of the piezoelectric body. In particular, in the case of receiving a voltage, which is output from the piezoelectric body due to the displacement of the flexible film, using a receiving circuit provided on the wiring substrate, it is possible to improve the reception accuracy since a high voltage signal is output from the piezoelectric body.

In the piezoelectric module according to the application example, it is preferable that the wiring substrate includes a polarization circuit that performs polarization processing by applying an electric field of 10 kV/cm or more between the first and second electrodes.

In the application example with this configuration, polarization processing of the piezoelectric body is performed by applying an electric field of 10 kV/cm or more between the first and second electrodes. In the application example with this configuration, for example, compared with a configuration in which a film-shaped piezoelectric body is interposed between a pair of electrodes along the thickness direction, the distance between the first and second electrodes is increased. Accordingly, it is not possible to perform appropriate polarization processing with the electric field less than 10 kV/cm. In contrast, by applying an electric field of 10 kV/cm or more between the first and second electrodes, it is possible to appropriately perform the polarization of the piezoelectric body.

An electronic apparatus according to one application example of the invention includes: a piezoelectric element including a flexible film, a piezoelectric body having a first surface in contact with the flexible film and a second surface opposite to the first surface, a first electrode provided between the first and second surfaces of the piezoelectric body, and a second electrode that is provided between the first and second surfaces of the piezoelectric body and that faces the first electrode with a first gap interposed therebetween in plan view as viewed from a thickness direction of the flexible film; and a control unit that controls the piezoelectric element.

The electronic apparatus according to the application example includes the piezoelectric element described above and the control unit that controls the piezoelectric element. Therefore, as in the application examples described above, it is possible to enhance the piezoelectric characteristics of the piezoelectric body. In particular, in the electronic apparatus that performs predetermined processing by detecting a voltage output from the piezoelectric body due to the displacement of the flexible film, a high voltage signal is output from the piezoelectric body. Accordingly, since the voltage detection accuracy is high, it is possible to improve the processing accuracy of the electronic apparatus.

A piezoelectric element manufacturing method according to one application example of the invention includes: forming, on a flexible film, a first electrode and a second electrode, which faces the first electrode with a first gap interposed therebetween in plan view as viewed from a thickness direction of the flexible film; and forming a piezoelectric body, which covers a part of the first electrode and a part of the second electrode, on the flexible film.

In this application example, since the first and second electrodes are formed before forming the piezoelectric body, it is possible to suppress the deterioration of the piezoelectric body due to electrode formation. Therefore, it is possible to easily manufacture the piezoelectric body having enhanced piezoelectric characteristics (high piezoelectric e constant).

A piezoelectric element manufacturing method according to one application example of the invention includes: forming a first piezoelectric layer on a flexible film; forming a first electrode and a second electrode, which faces the first electrode with a first gap interposed therebetween in plan view as viewed from a thickness direction of the flexible film, on the first piezoelectric layer; and forming a second piezoelectric layer, which covers apart of the first electrode and a part of the second electrode, on the first piezoelectric layer.

In this application example, the first piezoelectric layer that forms the piezoelectric body is formed on the flexible film, then the first and second electrodes are formed, and then the second piezoelectric layer that forms the piezoelectric body is formed. In this case, since the first and second electrodes are formed on the first piezoelectric layer, the piezoelectric characteristics of the first piezoelectric layer are degraded. However, the degradation of the piezoelectric characteristics of the second piezoelectric layer is suppressed. Accordingly, for example, compared with a case where the piezoelectric body is formed on the flexible film and the first and second electrodes are formed on the surface of the piezoelectric body, it is possible to manufacture the piezoelectric body having enhanced piezoelectric characteristics.

### BRIEF DESCRIPTION OF THE DRAWINGS

The invention will be described with reference to the accompanying drawings, wherein like numbers reference like elements.

FIG. 1 is a perspective view showing the schematic configuration of an ultrasonic measurement apparatus of a first embodiment.

FIG. 2 is a block diagram showing the schematic configuration of the ultrasonic measurement apparatus of the first embodiment.

FIG. 3 is a plan view showing the schematic configuration of an ultrasonic sensor in the first embodiment.

FIG. 4 is a plan view showing the schematic configuration of a transmission region of an element substrate in an ultrasonic device of the first embodiment.

FIG. 5 is a sectional view of the ultrasonic sensor taken along the line A-A in FIG. 4.

FIG. 6 is a plan view showing the schematic configuration of a receiving region of an element substrate in the ultrasonic device of the first embodiment.

FIG. 7 is a plan view showing the schematic configuration of a receiving transducer in the first embodiment.

FIG. 8 is a sectional view showing the schematic configuration of the ultrasonic sensor taken along the line A-A in FIG. 7.

FIG. 9 is a flowchart showing a method of manufacturing a receiving transducer in the first embodiment.

FIGS. 10A to 10E are diagrams schematically showing each step in the method of manufacturing a receiving transducer in the first embodiment.

FIG. 11 is a sectional view showing the schematic configuration of a receiving transducer in a second embodiment.

FIG. 12 is a flowchart showing a method of manufacturing a receiving transducer in the second embodiment.

FIGS. 13A to 13E are diagrams schematically showing each step in the method of manufacturing a receiving transducer in the second embodiment.

FIG. 14 is a sectional view showing the schematic configuration of a receiving transducer in a modification example of the second embodiment.

FIG. 15 is a plan view showing the schematic configuration of a receiving transducer in a third embodiment.

FIG. 16 is a sectional view showing the schematic configuration of the receiving transducer in the third embodiment.

FIG. 17 is a plan view showing the schematic configuration of a receiving transducer in a fourth embodiment.

FIG. 18 is a sectional view showing the schematic configuration of the receiving transducer in the fourth embodiment.

FIG. 19 is a plan view showing the schematic configuration of a modification example of a receiving transducer.

FIG. 20 is a diagram showing the measurement results of reception sensitivity in Examples 4 to 8 and Comparative Example 2.

### DESCRIPTION OF EXEMPLARY EMBODIMENTS

### First Embodiment

Hereinafter, an ultrasonic measurement apparatus as an electronic apparatus of a first embodiment according to the invention will be described with reference to the accompanying diagrams.

### Configuration of an Ultrasonic Measurement Aapparatus 1

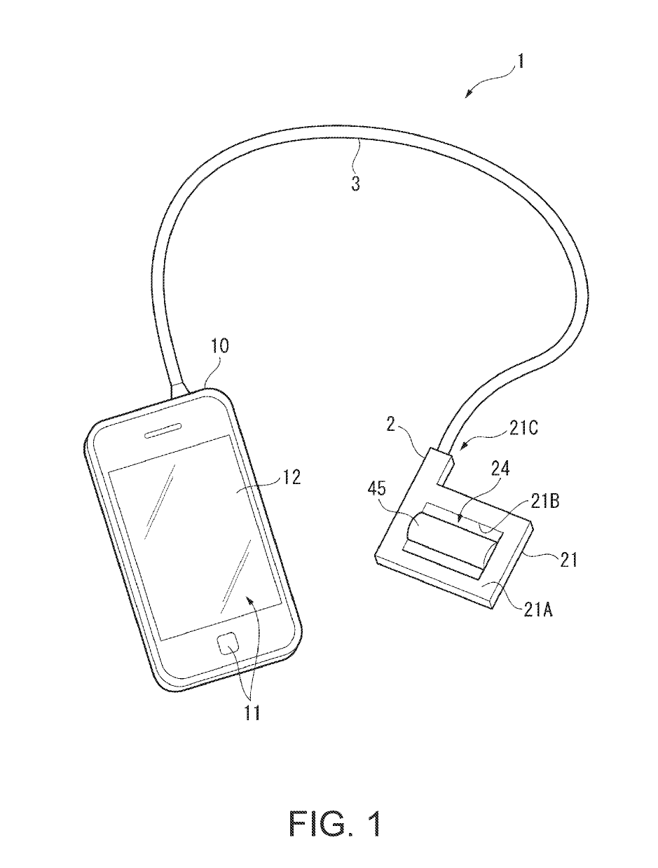

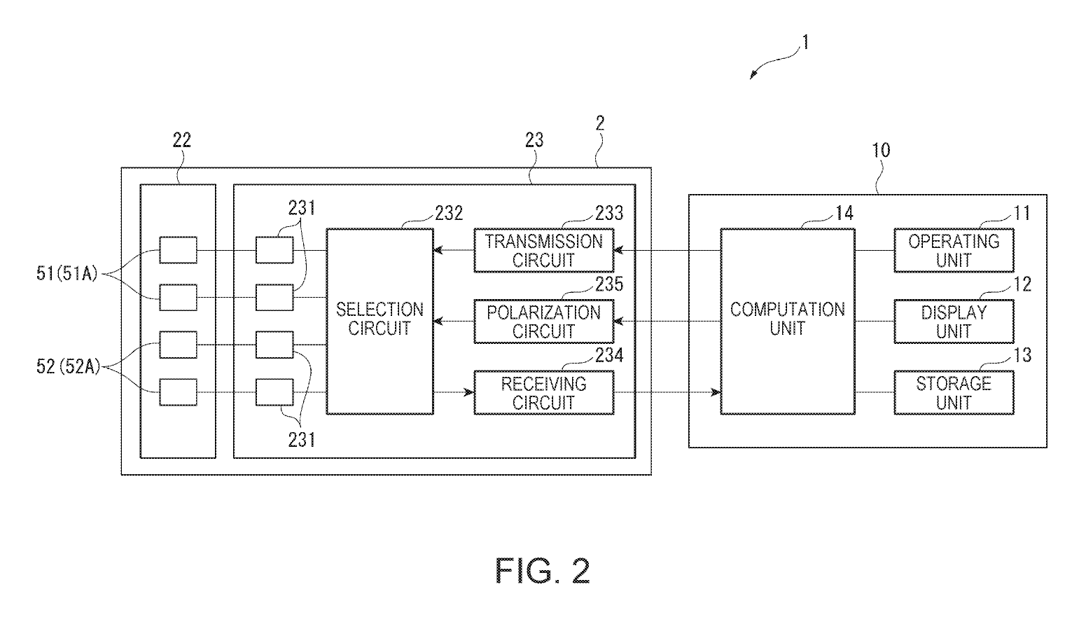

FIG. 1 is a perspective view showing the schematic configuration of the ultrasonic measurement apparatus 1 of the present embodiment. FIG. 2 is a block diagram showing the schematic configuration of the ultrasonic measurement apparatus 1.

The ultrasonic measurement apparatus 1 of the present embodiment corresponds to an electronic apparatus according to the invention. As shown in FIG. 1, the ultrasonic measurement apparatus 1 of the present embodiment includes an ultrasonic probe 2 and a control device 10 that is electrically connected to the ultrasonic probe 2 through a cable 3.

The ultrasonic probe 2 of the ultrasonic measurement apparatus 1 is brought into contact with the surface of the body (for example, a human body), and ultrasonic waves are emitted to the inside of the body from the ultrasonic probe 2. The ultrasonic probe 2 receives ultrasonic waves reflected by the organ in the body and, for example, acquires an internal tomographic image of the body or measures a state (for example, a blood flow) of the organ in the body based on the received signal.

### Configuration of the Ultrasonic Probe 2

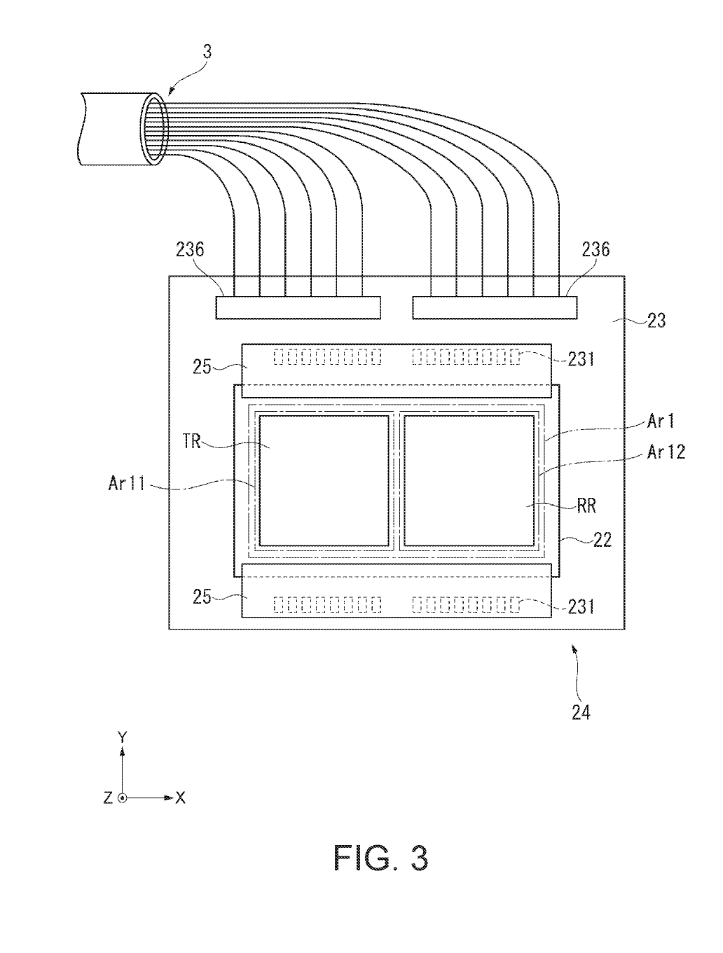

FIG. 3 is a plan view showing the schematic configuration of an ultrasonic sensor 24 in the ultrasonic probe 2.

The ultrasonic probe 2 includes a housing 21, an ultrasonic device 22 provided in the housing 21, and a wiring substrate 23 in which a driver circuit for controlling the ultrasonic device 22 and the like are provided. The ultrasonic sensor 24 is formed by the ultrasonic device 22 and the wiring substrate 23, and the ultrasonic sensor 24 forms a piezoelectric module according to the invention.

### Configuration of the Housing 21

As shown in FIG. 1, the housing 21 is formed in a rectangular box shape in plan view, for example. A sensor window 21B is provided on one surface (sensor surface 21A) perpendicular thereto in the thickness direction, so that a part of the ultrasonic device 22 is exposed. A passage hole 21C of the cable 3 is provided in a part of the housing 21 (in the example shown in FIG. 1, on a side surface), and the cable 3 is connected to the wiring substrate 23 in the housing 21 through the passage hole 21C. In addition, a gap between the cable 3 and the passage hole 21C is filled with, for example, a resin material. Accordingly, waterproofness is ensured.

In the present embodiment, an example of the configuration in which the ultrasonic probe 2 and the control device 10 are connected to each other using the cable 3. However, without being limited thereto, for example, the ultrasonic probe 2 and the control device 10 may be connected to each other by wireless communication, and various components of the control device 10 maybe provided in the ultrasonic probe 2.

### Configuration of the Ultrasonic Device 22

As shown in FIG. 3, the ultrasonic device 22 has an array region Arl where a transmission array TR for transmitting ultrasonic waves and a receiving array RR for receiving ultrasonic waves are formed. In FIG. 3, the transmission array TR and the receiving array RR have approximately the same array area. However, without being limited thereto, for example, the receiving array RR may be formed in a smaller size than the transmission array TR. The arrangement positions of the transmission array TR and the receiving array RR are not limited to the example shown in FIG. 3. For example, it is possible to adopt a configuration in which the receiving array RR is provided in a part of the transmission array TR or a configuration in which the transmission array TR and the receiving array RR are alternately arranged along an X direction (scanning direction).

The transmission array TR is configured by arranging a plurality of ultrasonic transmitting transducers 51 (hereinafter, abbreviated as transmitting transducers 51) that transmit ultrasonic waves in the shape of an array. In addition, the receiving array RR is configured by arranging a plurality of ultrasonic receiving transducers 52 (hereinafter, abbreviated as receiving transducers 52) that receive ultrasonic waves in the shape of an array. In the ultrasonic device 22 configured as described above, ultrasonic waves are transmitted from the transmission array TR, and reflected waves reflected by a measurement target are received by the receiving array RR.

In the following explanation, it is assumed that the scanning direction of the transmission array TR having a one-dimensional array structure, which will be described later, is an X direction and a slice direction perpendicular to the scanning direction is a Y direction.

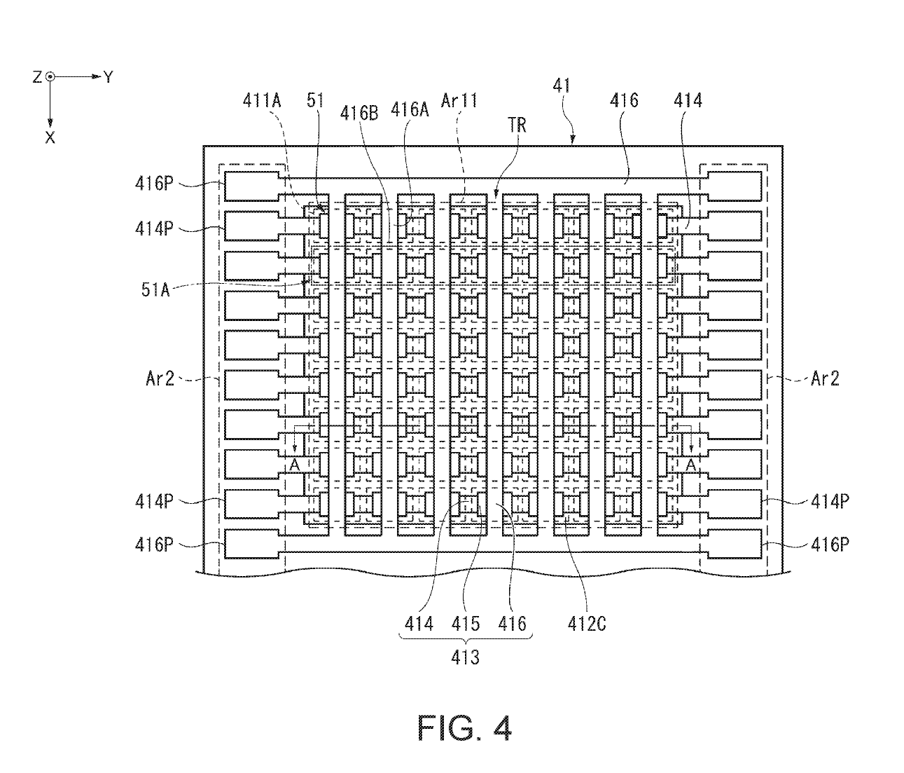

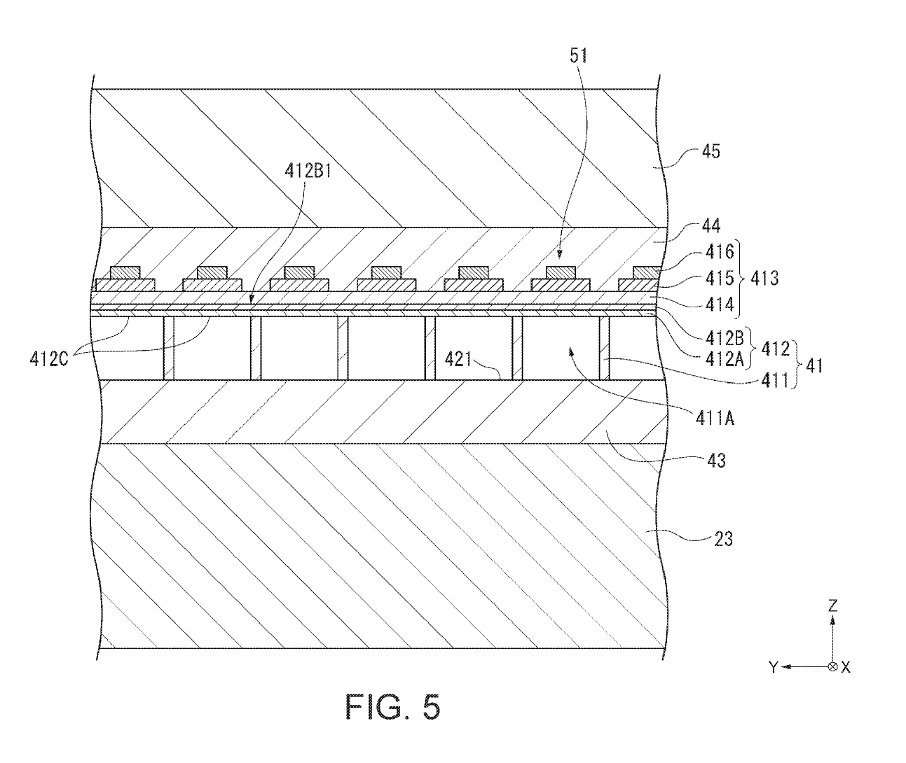

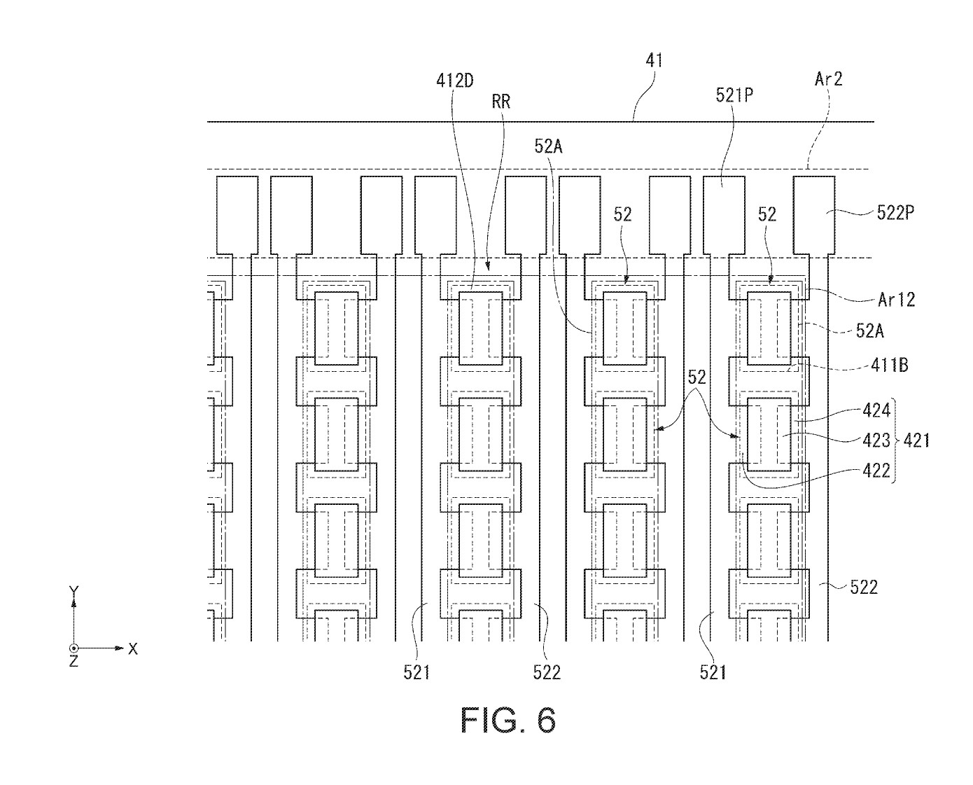

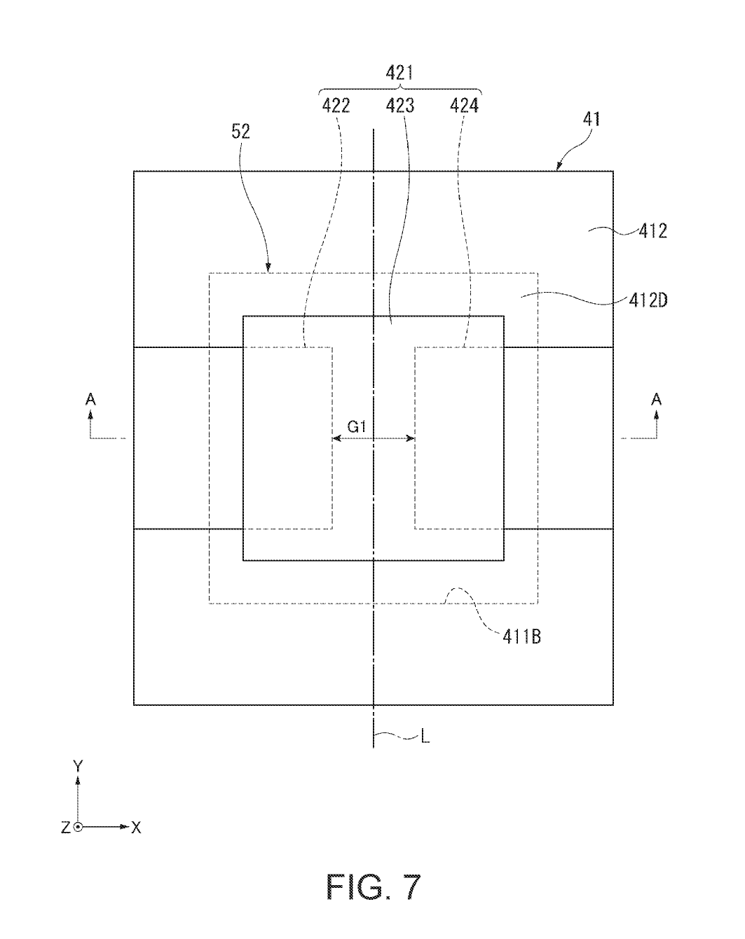

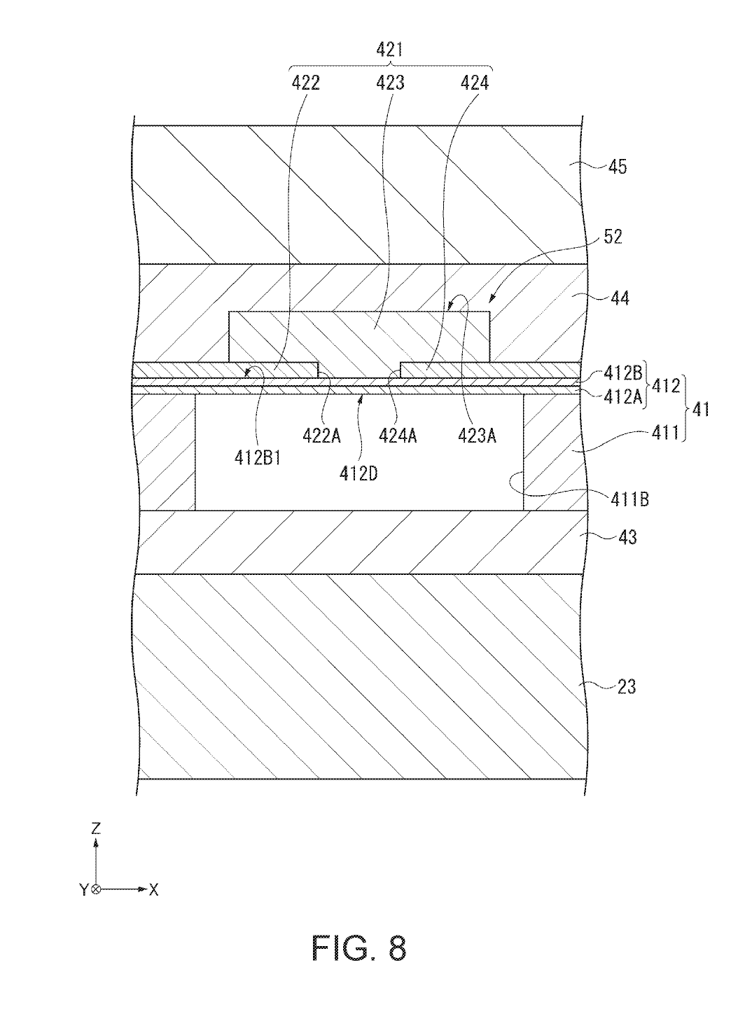

FIG. 4 is a plan view when an element substrate 41 in the transmission array TR of the ultrasonic device 22 is viewed from the opposite side (operation surface side) to a sealing plate 43. FIG. 5 is a sectional view of the ultrasonic sensor 24 taken along the line A-A in FIG. 4. FIG. 6 is a diagram schematically showing the configuration of the receiving array RR. FIG. 7 is a plan view schematically showing the receiving transducer 52 when viewed from the operation surface side of the element substrate 41. FIG. 8 is a schematic sectional view taken along the line A-A in FIG. 7.

As shown in FIGS. 5 and 8, the ultrasonic device 22 forming the ultrasonic sensor 24 is configured to include the element substrate 41, the sealing plate 43, an acoustic matching layer 44, and an acoustic lens 45 (refer to FIG. 1). In the present embodiment, as shown in FIGS. 5 to 8, in the transmission array TR and the receiving array RR, the element substrate 41, the sealing plate 43, the acoustic matching layer 44, and the acoustic lens 45 are common.

In the present embodiment, the array region Arl of the element substrate 41 includes a transmission region Ar11 and a receiving region Ar12. In the transmission region Ar11, a plurality of transmitting transducers 51 (refer to FIGS. 4 and 5) are arranged in the shape of an array to form the transmission array TR. In the receiving region Ar12, a plurality of receiving transducers 52 (refer to FIGS. 6, 7, and 8) are arranged in the shape of an array to form the receiving array RR. Hereinafter, the transmission array TR and the receiving array RR will be described in more detail.

### Configuration of the Transmission Array TR

As shown in FIG. 4, the transmission array TR is formed by a plurality of transmitting transducers 51 that are arranged in the shape of an array in the transmission region Ar11 of the element substrate 41.

In the transmission array TR, a transmitting transducer group 51A as one transmission channel is formed by a plurality of transmitting transducers 51 aligned in the Y direction (slice direction). In addition, in the transmission array TR, a plurality of transmitting transducer groups 51A are provided along the X direction (scanning direction) to form a one-dimensional array.

### Configuration of the Transmitting Transducer 51

As shown in FIG. 5, the transmitting transducer 51 is configured to include a part of the element substrate 41 and a driving element 413 provided on the element substrate 41.

The element substrate 41 includes a substrate body portion 411 and a support film 412 laminated on the substrate body portion 411. On the outside of the array region Arl of the element substrate 41, a terminal region Ar2 is provided so that the electrode line connected to each transmitting transducer 51 is lead out.

The substrate body portion 411 is, for example, a semiconductor substrate formed of Si. In the transmission region Ar11 of the substrate body portion 411, an opening 411A corresponding to each transmitting transducer 51 is provided. The size of the opening 411A is based on the frequency of the ultrasonic wave transmitted from the transmission array TR.

The support film 412 is provided on one surface of the substrate body portion 411 in order to close the opening 411A. A region of the support film. 412 that closes the opening 411A becomes a vibrating portion 412C that is vibrated in the thickness direction by the driving of the driving element 413 to be described later. By the vibration of the vibrating portion 412C, ultrasonic waves are output (transmitted). That is, a part of the element substrate 41 that forms the transmitting transducer 51 described above is the vibrating portion 412C of the support film 412 that closes the opening 411A, and the transmitting transducer 51 is formed by the vibrating portion 412C and the driving element 413.

More specifically, the support film 412 is a two-layer film, and is provided on a side of the substrate body portion 411 opposite to the sealing plate 43. The support film 412 includes a support layer 412A that closes the opening 411A and a surface layer 412B, which is provided on a side of the support layer 412A not facing the substrate body portion 411 and on which the driving element 413 is laminated. The support layer 412A is formed of, for example, SiO₂. In a case where the substrate body portion 411 is formed of Si and the support layer 412A is formed of SiO₂, it is possible to easily form the support layer 412A by performing thermal oxidation treatment on the one surface side of the substrate body portion 411.

The surface layer 412B is a layer that forms a first layer according to the invention, and is formed of a transition metal oxide. A surface of the surface layer 412B not facing the support layer 412A is a first surface 412B1 according to the invention.

The surface layer 412B is a layer on which parts of a lower electrode 414 and a piezoelectric film 415, which form the driving element 413, and a first electrode 422, a piezoelectric film 423, and a second electrode 424 that form a receiving element 421 as shown in FIGS. 6, 7, and 8 are laminated. Therefore, as the surface layer 412B, it is preferable to use a material having a high adhesion to the electrode materials and the piezoelectric material. Although will be described in detail later, in the receiving array RR, the piezoelectric film 423 interposed between the first and second electrodes 422 and 424 is laminated on the surface layer 412B. Therefore, as the surface layer 412B, it is preferable to use a film material that can prevent the diffusion of a high-vapor-pressure element, such as Pb contained in the piezoelectric film 423, when the piezoelectric film 423 is laminated and that can easily make the crystal orientation of the piezoelectric film 423 become the (100) orientation when the piezoelectric film. 423 is laminated. As the surface layer 412B, it is preferable to use a transition metal oxide. In particular, forming the surface layer 412B using ZrO₂ capable of easily suppressing the diffusion of Pb is more preferable. More specifically, if Ti having a thickness of 10 nm or less or BiFeTiO₃ having a thickness of 100 nm or less is laminated on ZrO₂ and then the piezoelectric film 423 is formed on the Ti or BiFeTiO₃, a piezoelectric body forming the piezoelectric film 423 is (100) preferentially oriented.

The driving element 413 is provided on the support film. 412 that closes each opening 411A, and includes the lower electrode 414, the piezoelectric film 415, and an upper electrode 416.

By applying a rectangular wave voltage of a predetermined frequency between the lower electrode 414 and the upper electrode 416 in the driving element 413, the piezoelectric film 415 expands or contracts in the in-plane direction. Since a surface of the piezoelectric film 415 facing the support film 412 is bonded to the support film 412 with the lower electrode 414 interposed therebetween, the amount of expansion and contraction of the surface of the piezoelectric film 415 facing the support film 412 is different from that of the opposite surface of the piezoelectric film 415. Accordingly, the piezoelectric film 415 vibrates by being displaced in the thickness direction due to the difference. By the vibration of the piezoelectric film 415, the vibrating portion 412C of the support film 412 also vibrates to transmit ultrasonic waves.

In the present embodiment, as shown in FIG. 4, a plurality of transmitting transducers 51 described above are provided along the X and Y directions in the transmission region Ar11 of the element substrate 41.

The lower electrode 414 is formed in a straight line along the Y direction, and is provided over a plurality of transmitting transducers 51 aligned along the Y direction. The transmitting transducer group 51A is formed by a plurality of transmitting transducers 51 that are connected to each other through the lower electrode 414 and are aligned in the Y direction (slice direction). The lower electrode 414 extends up to the terminal region Ar2. In the terminal region Ar2, a lower electrode terminal 414P provided at the end of the lower electrode 414 is electrically connected to the wiring substrate 23.

On the other hand, the upper electrode 416 includes an upper electrode body 416A, which is provided over a plurality of transmitting transducers 51 aligned along the X direction, and an upper electrode connecting portion 416B for connecting the ends of the upper electrode body 416A to each other. The end of the upper electrode connecting portion 416B extends up to the terminal region Ar2. In terminal region Ar2, an upper electrode terminal 416P provided at the end of the upper electrode connecting portion 416B is electrically connected to the wiring substrate 23.

### Configuration of the Receiving Array RR

As shown in FIG. 6, the receiving array RR is formed by a plurality of receiving transducers 52 that are arranged in the shape of an array in the receiving region Ar12 of the array region Arl of the element substrate 41. In the receiving array RR of the present embodiment, a receiving transducer group 52A as one receiving channel is formed by a plurality of receiving transducers 52, and a plurality of receiving transducer groups 52A are provided in the X direction.

As shown in FIG. 6, the receiving transducer group 52A includes a pair of electrode lines 521 and 522 provided along the Y direction and a plurality of receiving transducers 52 connected in parallel between the pair of electrode lines 521 and 522.

The electrode lines 521 and 522 are provided in a range from the receiving region Ar12 to the terminal region Ar2, and are electrically connected to the wiring substrate 23 through terminals 521P and 522P of the terminal region Ar2.

### Configuration of the Receiving Transducer 52

The receiving transducer 52 is a piezoelectric element according to the invention, and is configured to include a part of the element substrate 41 and the receiving element 421 laminated on the support film 412 of the element substrate 41.

As described above, in the present embodiment, in the transmission array TR and the receiving array RR, the element substrate 41 is a common member and is formed by the substrate body portion 411 and the support film 412.

In the receiving region Ar12 of the substrate body portion 411, an opening 411B corresponding to each receiving transducer 52 is provided as shown in FIGS. 6, 7, and 8. The opening 411B has a size corresponding to the frequency of the received ultrasonic wave. For example, in a case where ultrasonic waves are transmitted to a measurement target from the transmission array TR and the second harmonic wave reflected by the measurement target is received by the receiving array RR, the size of the opening 411B is smaller than the size of the opening 411A in the transmitting transducer 51.

In the same manner as in the transmitting transducer 51, the support film 412 closes the opening 411B. A region of the support film 412 that closes the opening 411B becomes a flexible portion 412D by being displaced when receiving ultrasonic waves. Thus, the region of the support film 412 that closes the opening 411B forms a flexible film according to the invention. When the flexible portion 412D is deformed, the receiving element 421 provided on the flexible portion 412D is also deformed, and an electrical signal is output from the receiving element 421. That is, a part of the element substrate 41 that forms the receiving transducer 52 described above is the flexible portion 412D of the support film 412 that closes the opening 411B, and the receiving transducer 52 is formed by the flexible portion 412D and the receiving element 421.

The receiving element 421 includes the first electrode 422, the piezoelectric film 423, and the second electrode 424.

As shown in FIG. 8, the first and second electrodes 422 and 424 are provided on the surface layer 412B of the support film 412.

The first and second electrodes 422 and 424 are formed of a conductive electrode material, such as Ir, Pt, IrOx, TiOx, SrRuO₃, and LaNiO₃. In this case, since the surface layer 412B of the support film 412 is formed of ZrO₂ that is a transition metal oxide, the electrode material can be appropriately brought into contact with the surface layer 412B.

The first electrode 422 is connected to an electrode line 521. In plan view as viewed along the Z direction (hereinafter, simply referred to as in “plan view”) as shown in FIGS. 6 and 7, the first electrode 422 is provided across the inside and outside of the opening 411B from the electrode line 521 to a predetermined position of the opening 411B on the −X side. In addition, the second electrode 424 is connected to an electrode line 522, and is provided across the inside and outside of the opening 411B from the electrode line 522 to a predetermined position of the opening 411B on the +X side in plan view.

The first and second electrodes 422 and 424 are axisymmetric with respect to a virtual line L (refer to FIG. 7) that passes through the center point of the opening 411B and is parallel to the Y direction.

A first end surface 422A that is an end surface of the first electrode 422 on the +X side is a plane that is located inside the opening 411B and is parallel to the Y direction. A second end surface 424A that is an end surface of the second electrode 424 on the −X side is a plane that is located inside the opening 411B and is parallel to the Y direction. That is, the first and second end surfaces 422A and 424A are parallel, and face each other with a gap G1 (first gap) interposed therebetween.

The piezoelectric film 423 corresponds to a piezoelectric body according to the invention. As shown in FIGS. 6, 7, and 8, the piezoelectric film 423 is provided on the flexible portion 412D so as to cover a region from a portion of the first electrode 422 including the first end surface 422A to a portion of the second electrode 424 including the second end surface 424A. In addition, between the first and second electrodes 422 and 424, the piezoelectric film 423 is in contact with the surface layer 412B of the flexible portion 412D. Therefore, in the present embodiment, the piezoelectric film 423 is filled in the gap G1 between the first and second electrodes 422 and 424. A surface (surface not facing the support film 412) of the piezoelectric film 423 is a second surface 423A according to the invention. That is, in the present embodiment, the first and second electrodes 422 and 424 are disposed between the first surface 412B1 and the second surface 423A.

It is preferable that the piezoelectric film 423 is formed of a perovskite type transition metal oxide. More preferably, the piezoelectric film 423 is formed of a perovskite type transition metal oxide containing Pb, Zr, and Ti. As the piezoelectric film 423, for example, lead zirconate titanate (PZT) can be mentioned.

The piezoelectric film 423 formed of such a perovskite type transition metal oxide (in particular, PZT) has particularly enhanced piezoelectric characteristics (high piezoelectric e constant), and the electrical signal output when the piezoelectric film. 423 is deformed is increased. The piezoelectric film. 423 is provided on the first electrode 422, the second electrode 424, and the surface layer 412B of flexible portion 412D. In this case, it is possible to easily make the crystal orientation of the piezoelectric film 423 become the (100) orientation. Also in this respect, it is possible to enhance the piezoelectric characteristics of the piezoelectric film 423. More specifically, Ti having a thickness of 10 nm or less or BiFeTiO₃ having a thickness of 100 nm or less is laminated on the first electrode 422, the second electrode 424, and the surface layer 412B of the flexible portion 412D, and then the piezoelectric film 423 is formed on the Ti or BiFeTiO₃. Then, the piezoelectric body (piezoelectric film 423) is (100) preferentially oriented.

### Characteristics of the Receiving Transducer 52

In the receiving transducer 52 including the receiving element 421 described above, when ultrasonic waves reflected by the measurement target are received by the flexible portion 412D, the flexible portion 412D vibrates. By the vibration of the flexible portion 412D, the receiving element 421 is also vibrated to deform the piezoelectric film 423. Then, electric charges move in response to the deformation (distortion) in the piezoelectric film 423, thereby generating a potential difference between the first and second electrodes 422 and 424. Accordingly, it is possible to detect the received ultrasonic waves by detecting the potential difference between the first and second electrodes 422 and 424.

Incidentally, the amount of deformation (the amount of distortion η) of the piezoelectric film 423 is generally proportional to a voltage V output from the piezoelectric body. Assuming that the electrostatic capacitance between the first and second electrodes 422 and 424 is C and the amount of charges in each of the electrodes 422 and 424 is Q, the following Equation (1) is satisfied.

V=Q/C (1)

Here, the amount of charges Q is expressed by the following Equation (2) using the area S of a region functioning as a capacitor in each of the electrodes 422 and 424 and the amount of charges per unit area (charge density) q. The electrostatic capacitance C is expressed by the following Equation (3) using a dielectric constant between the electrodes 422 and 424 (dielectric constant of the piezoelectric body) εand a distance d between the electrodes 422 and 424. In addition, assuming that the amount of displacement (the amount of distortion) of the piezoelectric film 423 is η and the piezoelectric constant (piezoelectric e constant) is e, the charge density q and the amount of distortion η are expressed by the following Equation (4). From Equations (1) to (4), the following Equation (5) can be derived.

Q=Sq (2)

C=Sc/d (3)

q=eη (4)

V=(de/ε)×η (5)

As expressed by the following Equation (1), the voltage V output from the piezoelectric film 423 when the flexible portion 412D is displaced increases as the electrostatic capacitance C decreases and the amount of charges Q increases. Therefore, it is possible to improve reception sensitivity when receiving ultrasonic waves. More specifically, as expressed by the Equations (2) to (5), it is possible to improve reception sensitivity by increasing the distance d between the electrodes 422 and 424, increasing the value of the piezoelectric e constant, and decreasing the dielectric constant ε.

In the present embodiment, the distance d between the first and second electrodes 422 and 424 is 2 μm or more and 8 μmor less. Usually, the piezoelectric film 423 is formed in a thickness of about 400 nm. That is, if the thickness of the piezoelectric film 423 is too large, the vibration of the flexible portion 412D is obstructed. Accordingly, good reception sensitivity cannot be obtained. If the thickness of the piezoelectric film 423 is too small, the piezoelectric characteristics of the piezoelectric film 423 are degraded since the influence of the missing of Pb (for example, in the case of PZT) is increased. From above, it is preferable that the piezoelectric film 423 is formed thin enough not to cause the degradation of the piezoelectric characteristics. Preferably, the piezoelectric film 423 is formed in a thickness of about 400 nm.

For example, if the piezoelectric film 423 is configured so as to be interposed between a pair of electrodes in the thickness direction, the distance d becomes the thickness of the piezoelectric film 423. Accordingly, since the distance d is a very small value, the output voltage V with respect to the amount of distortion 11 of the piezoelectric film 423 is reduced. That is, if the distance d between the electrodes is less than 2 μm, the sufficient output voltage V cannot be obtained from the piezoelectric film 423. Accordingly, the reception sensitivity of the receiving transducer 52 is reduced.

In contrast, in the present embodiment, the first and second electrodes 422 and 424 are disposed on the support film 412 as described above. Accordingly, it is possible to increase the distance between the electrodes 422 and 424. That is, the distance between the electrodes 422 and 424 can be set to be 2 μm or more and8 μm or less. Therefore, compared with the configuration in which the piezoelectric film 423 is interposed between a pair of electrodes in the thickness direction, it is possible to increase the voltage V output from the receiving element 421 (piezoelectric film 423).

By setting the distance d between the electrodes 422 and 424 to 8 μm or less, it is possible to improve the efficiency of polarization processing of a polarization circuit 235, which will be described later. That is, if the distance d between the electrodes 422 and 424 exceeds 8 μm, when performing the polarization processing of the piezoelectric film 423, it is necessary to increase a polarization voltage applied between the electrodes 422 and 424. In this case, since an expensive power supply needs to be used as a power supply provided in the polarization circuit 235, device cost is increased. In contrast, the polarization voltage at the time of polarization processing can be reduced by setting the distance d to 8 μm or less. That is, a low-cost power supply is used as a power supply provided in the polarization circuit 235. Accordingly, it is possible to reduce the device cost.

In addition, the piezoelectric film 423 of the present embodiment has enhanced piezoelectric characteristics (high piezoelectric e constant). Also in this respect, it is possible to increase the output voltage V when the piezoelectric film 423 is deformed. Therefore, it is possible to improve the reception sensitivity of the receiving transducer 52.

That is, in the present embodiment, since the piezoelectric film 423 is formed of PZT that is a perovskite type transition metal oxide, it is possible to enhance the piezoelectric characteristics. Ti having a thickness of 10 nm or less or BiFeTiO₃ having a thickness of 100 nm or less is laminated on the electrodes 422 and 424 or on the surface layer 412B formed of a transition metal oxide (ZrO₂), and then the piezoelectric film 423 is formed on the Ti or BiFeTiO₃. Accordingly, it is possible to easily make the crystal orientation of the piezoelectric film 423 become the (100) orientation. Also in this respect, it is possible to further enhance the piezoelectric characteristics of the piezoelectric film 423.

In the present embodiment, the first and second electrodes 422 and 424 are covered with the piezoelectric film 423. Since the receiving element 421 is obtained by forming the first and second electrodes 422 and 424 on the support film 412 and then forming the piezoelectric film 423, it is possible to suppress the deterioration of the piezoelectric film 423.

For example, in a case where the electrodes 422 and 424 are formed on the second surface 423A of the piezoelectric film 423, it is necessary to form the piezoelectric film 423 and then form an electrode material by sputtering or the like. Then, it is necessary to pattern the electrode material by etching processing (for example, ion milling). In this case, the piezoelectric film 423 is damaged at the time of sputtering of the electrode material. In addition, also at the time of patterning of the electrode material (at the time of etching processing), the piezoelectric film 423 is damaged. For this reason, defects occur, for example, in the crystal. This reduces the value of the piezoelectric e constant about tens of percent. In contrast, in the present embodiment, the piezoelectric film 423 is provided so as to cover the electrodes 422 and 424 as described above. Accordingly, also in the manufacturing process, the electrodes 422 and 424 are formed first, and then the piezoelectric film 423 is formed. Therefore, it is possible to suppress a reduction in the value of the piezoelectric e constant of the piezoelectric film 423 (degradation of the piezoelectric characteristics) without the piezoelectric film 423 being damaged when forming the electrodes 422 and 424.

Also in this respect, since the piezoelectric film 423 of the present embodiment has enhanced piezoelectric characteristics (high piezoelectric e constant), it is possible to further increase the output voltage V with respect to the amount of distortion η of the piezoelectric film 423.

### Configuration of the Sealing Plate 43, the Acoustic Matching Layer 44, and the Acoustic Lens 45

The sealing plate 43 is provided in order to reinforce the strength of the element substrate 41. For example, the sealing plate 43 is formed using a metal plate, such as a 42 alloy, or a semiconductor substrate, and is bonded to the element substrate 41. Since the material and thickness of the sealing plate 43 affect the frequency characteristics of the transmitting transducer 51 and the receiving transducer 52, it is preferable to set the material and thickness of the sealing plate 43 based on the center frequency of the ultrasonic wave transmitted and received.

As shown in FIGS. 5 and 8, the acoustic matching layer 44 is provided on the surface of the element substrate 41 not facing the sealing plate 43. Specifically, the acoustic matching layer 44 is filled between the element substrate 41 and the acoustic lens 45, and is formed in a predetermined thickness from the surface of the substrate body portion 411.

The acoustic lens 45 is provided on the acoustic matching layer 44, and is exposed to the outside from the sensor window 21B of the housing 21 as shown in FIG. 1.

Due to the acoustic matching layer 44 or the acoustic lens 45, ultrasonic waves transmitted from the transmitting transducer 51 efficiently propagate toward the body that is a measurement target, and ultrasonic waves reflected from the inside of the body efficiently propagate toward the receiving transducer 52. For this reason, the acoustic impedance of the acoustic matching layer 44 and the acoustic lens 45 is set to the intermediate acoustic impedance between the acoustic impedance of each of the transducers 51 and 52 of the element substrate 41 and the acoustic impedance of the body.

### Configuration of the Wiring Substrate 23

The ultrasonic device 22 is bonded to the wiring substrate 23, and a driver circuit or the like for controlling the transducers 51 and 52 is provided. As shown in FIG. 2, the wiring substrate 23 includes a terminal unit 231, a selection circuit 232, a transmission circuit 233, a receiving circuit 234, the polarization circuit 235, and a connector unit 236 (refer to FIG. 3).

Electrode lines (the lower electrode 414, the upper electrode 416, and the electrode lines 521 and 522) lead out to the terminal region Ar2 of the element substrate 41 are electrically connected to the terminal unit 231, for example, through a flexible printed circuit (FPC) 25 (refer to FIG. 3) when the ultrasonic device 22 is bonded to the wiring substrate 23. Each electrode line and the terminal unit 231 are connected to each other through the FPC 25.

In the present embodiment, the terminal unit 231 to which the upper electrode 416, which is a common electrode of each transmitting transducer 51, is connected is connected to, for example, a ground circuit, and the upper electrode 416 is set to have a predetermined common potential (for example, 0 potential).

In the present embodiment, the terminal unit 231 to which one of the electrode lines 521 and 522 connected to the receiving transducer 52, for example, the electrode line 522, is connected is connected to, for example, a ground circuit, and is set to have a common potential (for example, 0 potential).

The selection circuit 232 switches a transmission connection for connecting the ultrasonic sensor 24 and the transmission circuit 233 and a reception connection for connecting the ultrasonic sensor 24 and the receiving circuit 234 based on the control of the control device 10.

When switching to the transmission connection has been made by the control of the control device 10, the transmission circuit 233 outputs a transmission signal, which indicates the transmission of ultrasonic waves, to the ultrasonic sensor 24 through the selection circuit 232.

When switching to the reception connection has been made by the control of the control device 10, the receiving circuit 234 outputs a received signal, which is input from the ultrasonic sensor 24 through the selection circuit 232, to the control device 10. The receiving circuit 234 is configured to include, for example, a low noise amplifier circuit, a voltage controlled attenuator, a programmable gain amplifier, a low pass filter, and an A/D converter. The receiving circuit 234 performs various kinds of signal processing, such as the conversion of a received signal to a digital signal, removal of noise components, and amplification to a desired signal level, and then outputs the received signal after the processing to the control device 10.

The polarization circuit 235 performs polarization processing on the piezoelectric film 415 of the driving element 413 by applying a first polarization voltage between the lower electrode terminal 414P and the upper electrode terminal 416P.

In addition, the polarization circuit 235 performs polarization processing on the piezoelectric film 423 of the receiving element 421 by applying a second polarization voltage between the terminals 521P and 522P.

In the present embodiment, since the gap G1 between the first and second electrodes 422 and 424 of the receiving transducer 52 is large, the second polarization voltage should be larger than the first polarization voltage in order to enhance the piezoelectric characteristics of the piezoelectric film 423 sufficiently. The second polarization voltage is set such that an electric field of 10 kV/cm or more is applied between the first and second electrodes 422 and 424 of each receiving transducer 52.

In the present embodiment, the distance d between the electrodes 422 and 424 is d=6 μm, and 30 V is applied as the second polarization voltage. Accordingly, the electric field of 500 kV/cm is applied to the piezoelectric film 423 of each receiving transducer 52.

The connector unit 236 is connected to the transmission circuit 233 and the receiving circuit 234. In addition, the cable 3 is connected to the connector unit 236. As described above, the cable 3 is lead out from the passage hole 21C of the housing 21 to be connected to the control device 10.

### Configuration of the Control Device 10

As shown in FIG. 2, the control device 10 is configured to include, for example, an operating unit 11, a display unit 12, a storage unit 13, and a computation unit 14. As examples of the control device 10, a terminal device, such as a tablet terminal, a smartphone, or a personal computer, may be used, or a dedicated terminal device for operating the ultrasonic probe 2 may be used.

The operating unit 11 is a user interface (UI) used when the user operates the ultrasonic measurement apparatus 1. For example, the operating unit 11 can be configured to include a touch panel provided on the display unit 12, operation buttons, a keyboard, a mouse, or the like.

The display unit 12 is formed using, for example, a liquid crystal display, and displays an image thereon.

The storage unit 13 stores various programs and various kinds of data for controlling the ultrasonic measurement apparatus 1.

The computation unit 14 is configured to include, for example, an arithmetic circuit, such as a central processing unit (CPU), and a storage circuit, such as a memory. The computation unit 14 reads various programs stored in the storage unit 13 and executes the various programs, thereby performing the generation of a transmission signal and the control of output processing for the transmission circuit 233 and performing received signal frequency setting, gain setting, or the like for the receiving circuit 234.

The computation unit 14 controls the polarization circuit 235 to perform polarization processing of the piezoelectric film 415 of the transmitting transducer 51 and the piezoelectric film. 423 of the receiving transducer 52. As a timing for performing the polarization processing, for example, the polarization processing may be performed each time ultrasonic measurement is performed or may be performed every predetermined time (for example, every hour) as well as performing the polarization processing at the time of shipping.

### Method of Manufacturing the Receiving Transducer 52

Next, a method of manufacturing the receiving transducer 52 will be described.

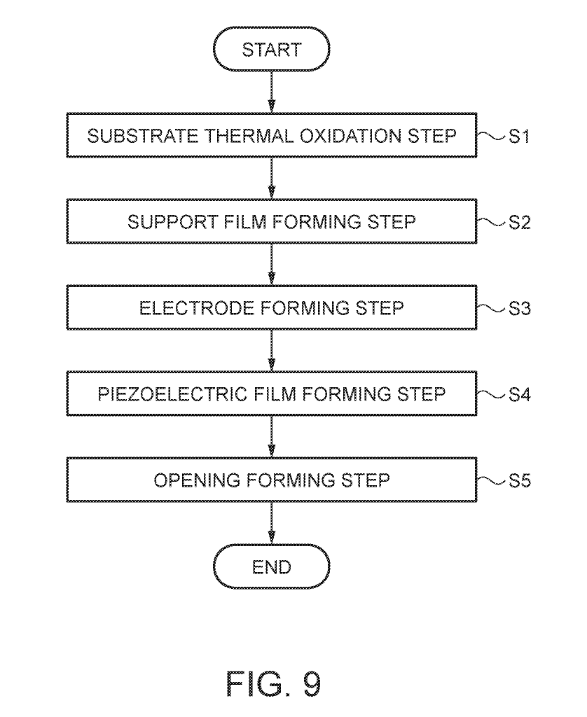

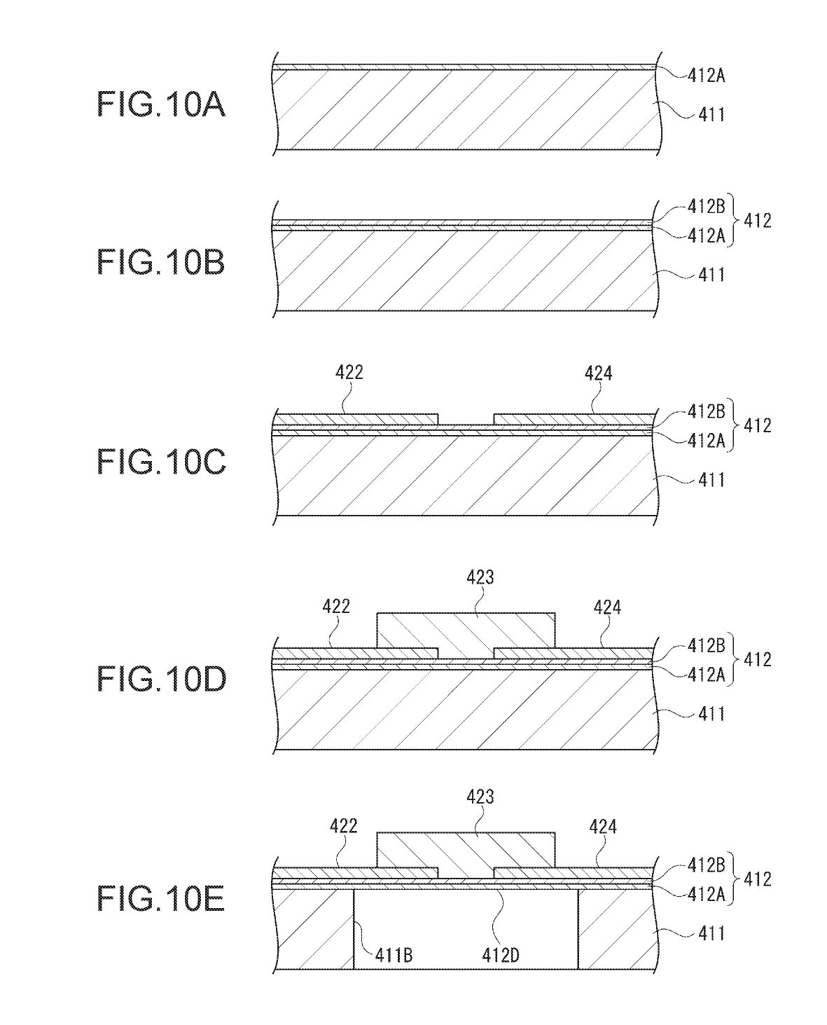

FIG. 9 is a flowchart showing a method of manufacturing the receiving transducer 52 of the present embodiment. FIGS. 10A to 10E are diagrams schematically showing each step in the method of manufacturing the receiving transducer 52.

In the manufacturing of the receiving transducer 52, thermal oxidation processing is first performed on one surface of the substrate body portion 411 formed of S1 (step S1 in FIG. 9: substrate thermal oxidation step). In step S1, as shown in FIG. 10A, Si of the surface of the substrate body portion 411 is oxidized to become SiO₂. As a result, the support layer 412A of the support film 412 is formed.

Then, as shown in FIG. 10B, the surface layer 412B is formed on the support layer 412A, thereby forming the support film 412 (step S2 in FIG. 9: support film forming step). Specifically, the surface layer 412B formed of ZrO₂ is formed by forming a Zr layer on the support layer 412A formed in step S1 using, for example, sputtering and performing thermal oxidation processing on the Zr layer.

Then, as shown in FIG. 10C, the first and second electrodes 422 and 424 are formed on the support film 412 (step S3 in FIG. 9: electrode forming step). For example, the first and second electrodes 422 and 424 are formed by forming an electrode material using sputtering and patterning the electrode material by etching processing or the like. As examples of the electrode material, as described above, Ir, Pt, IrOx, TiOx, SrRuO₃, and LaNiO₃ can be used. In the present embodiment, Pt is used.

More specifically, Ti having a thickness of 10 nm or less or BiFeTiO₃ having a thickness of 100 nm or less is laminated on the first electrode 422, the second electrode 424, and the surface layer 412B of the flexible portion 412D. In this case, when forming the piezoelectric film 423 in a piezoelectric body forming step to be described later, the piezoelectric body forming the piezoelectric film 423 is (100) preferentially oriented.

Then, as shown in FIG. 10D, the piezoelectric film 423 is formed (step S4 in FIG. 9: piezoelectric film forming step (piezoelectric body forming step)).

In step S4, PZT is formed using a solution method, for example. Here, the composition ratio of components in the PZT is preferably Zr:Ti=52:48. With such a composition, it is possible to further improve the piezoelectric characteristics of the piezoelectric film 423. In the formation of the PZT using a solution method, a PZT solution is coated on the surface layer 412B, the first electrode 422, and the second electrode 424 (coating step). Then, the coated PZT solution is baked (baking step). In the baking step, the coated PZT solution is baked under the conditions of, for example, prebaking at 400° C. and RTA baking at 700° C.

In this case, as described above, since the PZT is formed on the electrodes 422 and 424 formed of Pt or the surface layer 412B formed of ZrO₂, it becomes easy to make the crystal orientation of the PZT become the (100) orientation.

In addition, the coating step and the baking step are performed repeatedly multiple times. As a result, a piezoelectric film having a desired thickness is formed.

Then, the formed piezoelectric film is patterned by etching processing (ion milling), thereby forming the piezoelectric film 423 as shown in FIG. 10D.

Then, by performing etching processing on the surface of the substrate body portion 411 not facing the support film 412, thereby forming the opening 411B in the substrate body portion 411 as shown in FIG. 10E (step S5 in FIG. 9: opening forming step). In step S5, the substrate body portion 411 formed of Si is etched using the support layer 412A, which is formed of SiO₂, of the support film 412 as an etching stopper.

In such a manner described above, the receiving transducer 52 is formed.

### Effects of the First Embodiment

The ultrasonic measurement apparatus 1 of the present embodiment includes the ultrasonic probe 2, and the ultrasonic sensor 24 formed by the wiring substrate 23 and the ultrasonic device 22 is provided in the ultrasonic probe 2. The ultrasonic device 22 includes the receiving array RR in which a plurality of receiving transducers 52 for receiving ultrasonic waves are provided. The receiving transducer 52 includes the flexible portion 412D, the first electrode 422 provided on the flexible portion 412D, the second electrode 424 that is provided on the flexible portion 412D and that faces the first electrode 422 with the gap G1 interposed therebetween in plan view, and the piezoelectric film 423 that covers a portion including the first end surface 422A of the first electrode 422 and the second end surface 424A of the second electrode 424.

The receiving transducer 52 is formed by forming the first and second electrodes 422 and 424 before the formation of the piezoelectric film 423 and then forming the piezoelectric film 423. Accordingly, since the degradation of the piezoelectric characteristics of the piezoelectric film 423 at the time of electrode formation does not occur, it is possible to enhance the piezoelectric characteristics, for example, compared with a configuration in which the electrodes 422 and 424 are provided on the second surface 423A of the piezoelectric film 423. For this reason, it is possible to improve the reception sensitivity in each receiving transducer 52. As a result, when transmitting ultrasonic waves from the transmission array TR and receiving the reflected ultrasonic waves, which are reflected by the measurement target, at the receiving array RR, it is possible to accurately detect the reception timing of the ultrasonic wave and the intensity of the reflected ultrasonic waves.

In addition, since the piezoelectric film 423 is interposed between the first and second electrodes 422 and 424, it is possible to suppress the dielectric breakdown of the piezoelectric film 423.

In general, in the receiving element 421 in which the electrodes 422 and 424 are in contact with the piezoelectric film 423, a nano-scale void or tunnel structure is present between the electrodes 422 and 424 and the piezoelectric film 423. For example, in the case of a configuration in which the first and second electrodes 422 and 424 are provided on the second surface 423A of the piezoelectric film 423, H₂O molecules in the atmosphere are diffused into the boundary plane between the electrodes 422 and 424 and the piezoelectric film 423 through a void or a tunnel structure at the time of polarization processing.

In this case, H₂O molecules cause electrolysis on the boundary plane due to the influence of the applied pulse voltage that fluctuates in the positive and negative directions. As a result, since generated H groups or OH groups are attached to the nano-scale crack surface present in the piezoelectric film 423, the cracking of the piezoelectric film 423 is allowed to proceed. This causes a dielectric breakdown. Assuming that the perovskite type transition metal oxide forming the piezoelectric film 423 is ABO₃, H groups are adsorbed onto the A site and OH groups are adsorbed onto the B site to become stabilized, thereby accelerating the progress of cracking.

In contrast, in the present embodiment, since the piezoelectric film 423 is interposed between the first and second electrodes 422 and 424, H₂O molecules in the atmosphere do not enter the boundary plane. Accordingly, since it is possible to suppress the occurrence of dielectric breakdown as described above, it is possible to maintain high reception sensitivity for a long period of time. As a result, it is possible to enhance the reliability of the receiving transducer 52.

In the present embodiment, the first and second electrodes 422 and 424 are spaced apart from each other with a gap of 2 μm or more and 8 μm or less interposed therebetween. In such a configuration, in the polarization processing of the piezoelectric film 423, a high polarization voltage is required compared with a configuration in which the piezoelectric film 415 is interposed between the lower electrode 414 and the upper electrode 416 in the thickness direction, such as the configuration of the transmitting transducer 51. Therefore, for example, in the case of a configuration in which the first and second electrodes 422 and 424 are provided on the second surface 423A of the piezoelectric film 423, discharge occurs in the air between the first and second electrodes 422 and 424. As a result, there is a case in which the polarization processing of the piezoelectric film 423 is not sufficiently performed. In contrast, in the present embodiment, as described above, an air layer is not interposed between the first and second electrodes 422 and 424, and the piezoelectric film 423 is interposed between the first and second electrodes 422 and 424. Accordingly, since the above-described discharge does not occur, it is possible to appropriately perform the polarization processing of the piezoelectric film 423 during the polarization processing.

In the receiving transducer 52 of the present embodiment, the first and second electrodes 422 and 424 are provided on the flexible portion 412D. That is, the first and second electrodes 422 and 424 are provided between the first surface 412B1, which is a surface of the surface layer 412B, and the piezoelectric film 423.

In this case, since the piezoelectric film 423 can be formed after forming the first and second electrodes 422 and 424 on the flexible portion 412D, the piezoelectric film 423 is not formed at the time of electrode formation. Accordingly, there is no deterioration of the piezoelectric film 423 at the time of electrode formation. As a result, since the deterioration of the piezoelectric film 423 is suppressed, it is possible to further enhance the piezoelectric characteristics.

In the present embodiment, the first end surface 422A of the first electrode 422 and the second end surface 424A of the second electrode 424 are parallel.

In general, in a case where there is a potential difference between electrodes facing each other, electric charges move to a position where the distance between the electrodes is the shortest. In the present embodiment, when the piezoelectric film 423 is deformed, electric charges are held in a second-half range where the first end surface 422A and the second end surface 424A face each other. Therefore, it is possible to improve the voltage detection accuracy (it is possible to improve reception sensitivity).

In the present embodiment, the piezoelectric film 423 is formed of PZT that is a perovskite type transition metal oxide. The perovskite type transition metal oxide as a piezoelectric material has enhanced piezoelectric characteristics. Among the perovskite type transition metal oxides, the PZT has enhanced piezoelectric characteristics (high piezoelectric e constant) in particular. Therefore, as expressed by Equation (5), it is possible to increase the voltage V that is output from the piezoelectric film 423 when the flexible portion 412D is displaced. As a result, it is possible to improve reception sensitivity in the receiving transducer 52.

In the present embodiment, the support film 412 forming the flexible portion 412D includes the surface layer 412B in contact with the piezoelectric film 423, and the surface layer 412B is formed of ZrO₂ that is a transition metal oxide. When forming the piezoelectric film 423, such as PZT, on the surface of the transition metal oxide (in particular, ZrO₂), the crystal orientation of the piezoelectric film 423 easily becomes the (100) orientation. Therefore, since it is possible to further enhance the piezoelectric characteristics of the piezoelectric film 423 by providing the surface layer 412B, it is possible to further improve the reception sensitivity of the receiving transducer 52.

In the present embodiment, the gap G1 between the first and second electrodes 422 and 424 is set to the distance d of 2 μm or more and 8 μm less. In a case where the distance of the gap G1 is less than 2 μm, the output voltage V with respect to the amount of distortion η of the piezoelectric film 423 is reduced. Accordingly, reception sensitivity is reduced. In a case where the distance of the gap G1 is larger than 8 m, it is necessary to apply a larger voltage as a second polarization voltage at the time of polarization processing. Accordingly, since a power supply used in the polarization circuit 235 becomes expensive, device cost is increased. In contrast, by using the gap G1 described above, it is possible to sufficiently increase the output voltage V with respect to the amount of distortion η of the piezoelectric film 423 and to keep the second polarization voltage at the time of polarization processing in a practical range.

The wiring substrate 23 of the present embodiment includes the polarization circuit 235, which applies a second polarization voltage, between the first and second electrodes 422 and 424 of the receiving transducer 52. The polarization circuit 235 applies an electric field of 10 kV/cm or more, as the second polarization voltage, between the first and second electrodes 422 and 424 of the receiving transducer 52.

As described above, in the present embodiment, the gap G1 between the first and second electrodes 422 and 424 is a distance of 2 m or more and 8 μm or less. Accordingly, if the second polarization voltage is an electric field less than 10 kV/cm, it is not possible to appropriately perform the polarization processing of the piezoelectric film 423. In contrast, by applying an electric field of 10 kV/cm or more between the first and second electrodes, it is possible to appropriately perform the polarization of the piezoelectric body.

### Second Embodiment

Next, a second embodiment of the invention will be described.

In the first embodiment, an example is shown in which the first and second electrodes 422 and 424 are formed on the first surface 412B1 of the support film 412. In contrast, in the second embodiment, positions where the first and second electrodes 422 and 424 are formed are different from those in the first embodiment.

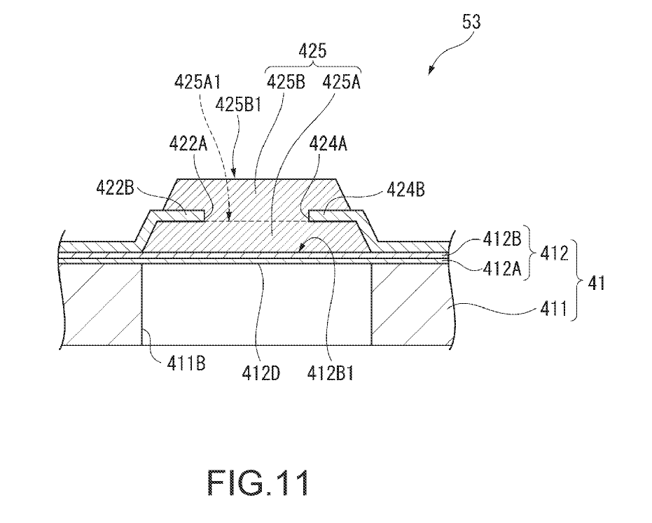

FIG. 11 is a sectional view showing the schematic configuration of a receiving transducer in the second embodiment. In the following explanation, the same components as in the first embodiment are denoted by the same reference numerals, and the explanation thereof will be omitted or simplified.

In a receiving transducer 53 of the present embodiment, as shown in FIG. 11, first and second electrodes 422B and 424B are embedded in a piezoelectric film 425.

Specifically, the piezoelectric film 425 is formed by a first piezoelectric layer 425A laminated on a flexible portion 412D and a second piezoelectric layer 425B laminated on the first piezoelectric layer 425A.

The first and second electrodes 422B and 424B are provided between a first surface 412B1 of the flexible portion 412D and a second surface 425B1 of the piezoelectric film 425 that is a surface not facing the flexible portion 412D. That is, the first and second electrodes 422B and 424B are formed on the surface (third surface 425A1) of the first piezoelectric layer 425A facing the second piezoelectric layer 425B between the first and second piezoelectric layers 425A and 425B.

In addition, the second piezoelectric layer 425B is interposed between a gap G1 between the first and second electrodes 422B and 424B. That is, an air layer is not interposed between the first and second electrodes 422B and 424B as in the first embodiment.

### Method of Manufacturing the Receiving Transducer 53

Next, a method of manufacturing the receiving transducer 53 will be described.

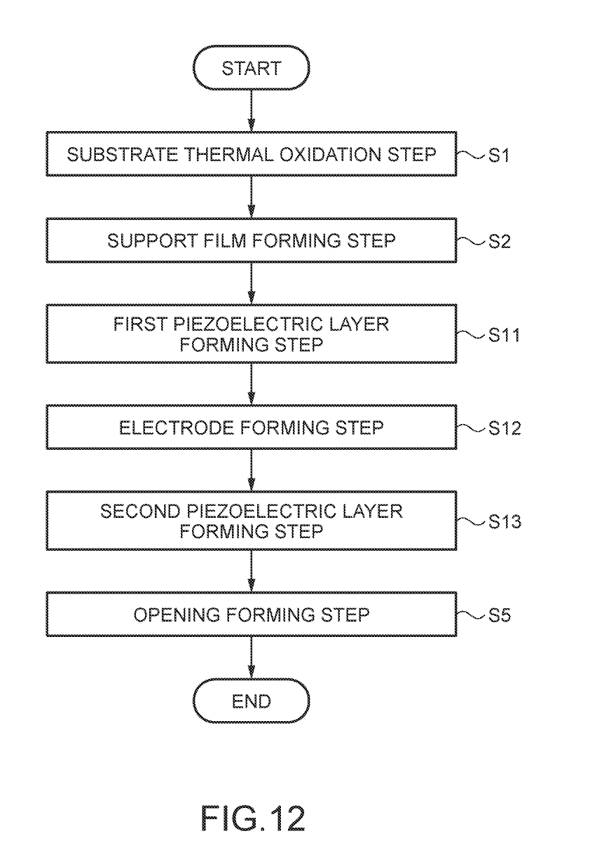

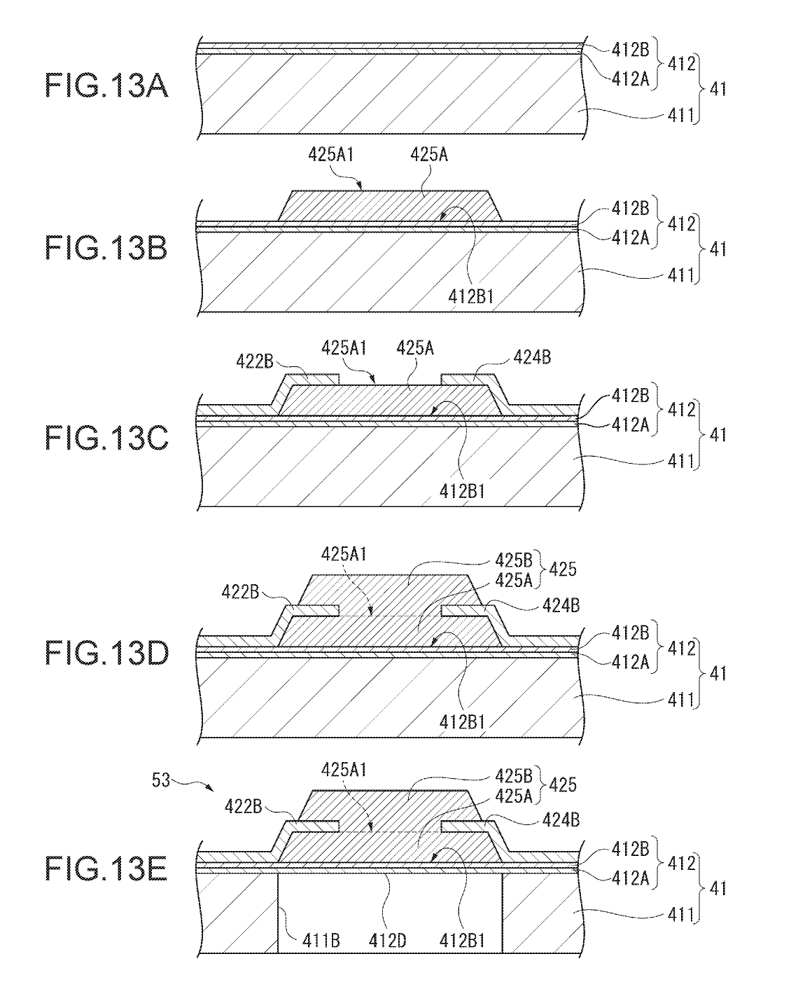

FIG. 12 is a flowchart showing a method of manufacturing the receiving transducer 53. FIGS. 13A to 13E are diagrams schematically showing each step in the method of manufacturing the receiving transducer 52.

In the method of manufacturing the receiving transducer 53 of the present embodiment, as shown in FIG. 12, the same steps S1 and S2 as in the first embodiment are performed to form the element substrate 41 as shown in FIG. 13A.

Then, in the present embodiment, the first piezoelectric layer 425A is formed (step S11: first piezoelectric layer forming step). In step S11, the first piezoelectric layer 425A is formed on the first surface 412B1 of the surface layer 412B. In this case, since the surface layer 412B is formed of ZrO2 that is a transition metal oxide, it is possible to make the crystal orientation of the first piezoelectric layer 425A become the (100) orientation in the same manner as for the piezoelectric film 423 of the first embodiment. More specifically, Ti having a thickness of 10 nm or less or BiFeTiO₃ having a thickness of 100 nm or less is laminated on the surface layer 412B of the flexible portion 412D formed of ZrO₂, and the first piezoelectric layer 425A is formed thereon. Accordingly, the piezoelectric body forming the first piezoelectric layer 425A is (100) preferentially oriented.

Formation of the first piezoelectric layer 425A is the same as the formation of the piezoelectric film 423 in the first embodiment. For example, by repeating the PZT solution coating step and the PZT solution baking step, a multi-layered PZT laminate is formed. Then, islands are formed by performing etching processing (ion milling) on the PZT laminate, thereby forming the first piezoelectric layer 425A as shown in FIG. 13B.

Then, the first and second electrodes 422B and 424B are formed (step S12: electrode forming step). In step S12, an electrode material is formed as a film in a region ranging from the third surface 425A1 among the surfaces of the first piezoelectric layer 425A to the support film 412 and is patterned by etching processing, thereby forming the first and second electrodes 422B and 424B as shown in FIG. 13C. More specifically, Ti having a thickness of 10 nm or less or BiFeTiO₃ having a thickness of 100 nm or less is laminated on the first electrode 422A, the second electrode 424B, and the upper surface of the first piezoelectric layer 425A. In this case, the piezoelectric body forming the second piezoelectric layer 425B that is formed in the second piezoelectric layer forming step of step S13, which will be described later, is (100) preferentially oriented.

Then, the second piezoelectric layer 425B is formed (step S13: second piezoelectric layer forming step). In step S13, the second piezoelectric layer 425B that covers a part of the first electrode 422B, a part of the second electrode 424B, and the first piezoelectric layer 425A is formed.

In step S13, for example, PZT solution coating step and PZT solution baking step are repeated using the same solution method as in step S11, thereby forming a multi-layered PZT laminate. Then, islands are formed by performing etching processing (ion milling) on the PZT laminate, thereby forming the first piezoelectric layer 425A as shown in FIG. 13D.

Then, as in step S5 of the first embodiment, the opening 411B is formed in the element substrate 41, thereby forming the flexible portion 412D. In such a manner described above, the receiving transducer 53 is manufactured.

Incidentally, in the present embodiment, in step S12, the first and second electrodes 422B and 424B are formed on the upper surface of the first piezoelectric layer 425A. Therefore, in step S12, the first piezoelectric layer 425A is deteriorated, and the piezoelectric characteristics are also degraded. However, since the second piezoelectric layer 425B formed in step S13 is formed after the electrode forming step, degradation of the piezoelectric characteristics of the second piezoelectric layer 425B is suppressed. Accordingly, for example, compared with a case where a pair of electrodes are provided on the second surface 425B1 of the piezoelectric film 425, degradation of the piezoelectric characteristics is suppressed.

In addition, if the coating step and the baking process using a PZT solution are repeatedly performed multiple times, Pb concentration on the lower layer side (flexible portion 412D side) of the piezoelectric film 425 becomes slightly lower than the Pb concentration on the upper layer side (second surface 425B1 side) due to the diffusion of Pb in the PZT. If the Pb concentration is low, the piezoelectric characteristics of the piezoelectric film 425 are degraded.

In contrast, in the present embodiment, the first and second electrodes 422B and 424B are formed on the third surface 425A1 during the formation of the second piezoelectric layer 425B. For this reason, during the formation of the second piezoelectric layer 425B, the diffusion of Pb of the second piezoelectric layer 425B into the first piezoelectric layer 425A is suppressed. Therefore, a Pb concentration distribution in the piezoelectric film 425 becomes more uniform than that in the piezoelectric film 423 of the first embodiment, for example. In this respect, it is possible to improve the piezoelectric characteristics of the piezoelectric film 425.

### Effects of the Second Embodiment

In the receiving transducer 53 of the present embodiment, the first and second electrodes 422B and 424B are embedded in the piezoelectric film 425.

In this case, since the first and second electrodes 422B and 424B are formed on the third surface 425A1 after forming the first piezoelectric layer 425A, the first piezoelectric layer 425A is deteriorated. However, since the second piezoelectric layer 425B is formed after the electrode forming step, the deterioration of the second piezoelectric layer 425B is suppressed.

In the second piezoelectric layer forming step, diffusion of atoms to the first piezoelectric layer 425A from the second piezoelectric layer 425B, which is newly formed, occurs. Therefore, crystal defects generated in the first piezoelectric layer 425A in the electrode forming step are repaired.

Accordingly, for example, compared with a case where electrodes are formed on the third surface 425A1 of the piezoelectric film 425, it is possible to enhance the piezoelectric characteristics of the piezoelectric film 425.

In addition, since the first and second electrodes 422B and 424B are formed on the surface (third surface 425A1) of the first piezoelectric layer 425A, the diffusion of Pb of the second piezoelectric layer 425B to the first piezoelectric layer 425A side is suppressed when forming the second piezoelectric layer 425B. Accordingly, the degradation of the piezoelectric characteristics of the second piezoelectric layer 425B is further suppressed.

That is, when the flexible portion 412D is displaced, the amount of distortion of the second piezoelectric layer 425B of the piezoelectric film 425 is larger than the amount of distortion of the first piezoelectric layer 425A. Accordingly, in a case where the first piezoelectric layer 425A is compared with the second piezoelectric layer 425B, it is preferable that the second piezoelectric layer 425B has more enhanced piezoelectric characteristics (higher piezoelectric e constant) than the first piezoelectric layer 425A. In the present embodiment, as described above, the degradation of the piezoelectric characteristics of the second piezoelectric layer 425B is more suppressed than that of the first piezoelectric layer 425A. Therefore, it is possible to improve the reception sensitivity of the receiving transducer 53.

In addition, in the present embodiment, the first and second electrodes 422B and 424B are provided on the same plane (on the third surface 425A1). In this case, since the first and second electrodes 422B and 424B can be simultaneously formed, it is possible to improve the manufacturing efficiency.

Modification examples of the second embodiment

In the second embodiment, the configuration is exemplified in which the second piezoelectric layer 425B covers the first and second electrodes 422B and 424B on the third surface 425A1. However, the information is not limited to thereto.

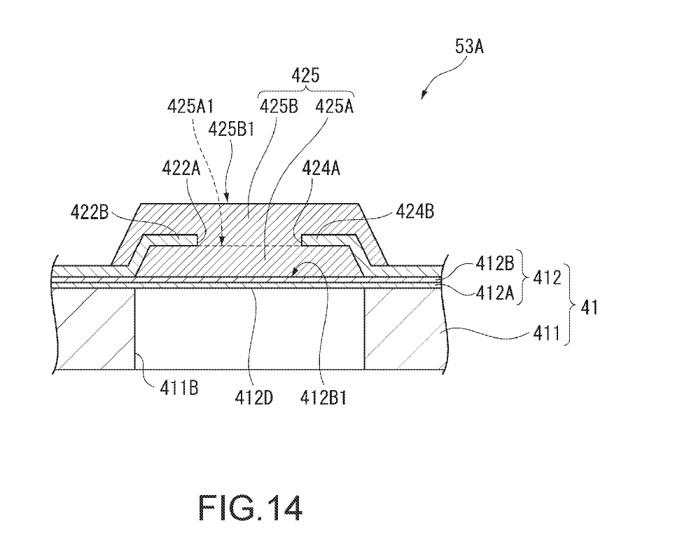

FIG. 14 is a sectional view showing the schematic configuration of a receiving transducer 53A in a modification example of the second embodiment.

As shown in FIG. 14, the second piezoelectric layer 425B may be formed so as to cover the entire regions, which are located on the first piezoelectric layer 425A, of the first and second electrodes 422B and 424B.

The end of the first piezoelectric layer 425A is tapered as shown in FIG. 14. Accordingly, in a case where an electrode material is formed as a film, for example, by sputtering or spin coating, the electrode thickness with respect to the tapered portion is reduced. In contrast, by forming the second piezoelectric layer 425B as in this modification example, it is possible to cover and protect an electrode portion formed on the tapered portion of the first piezoelectric layer 425A. Therefore, it is possible to prevent disconnection of the first electrode 422B or the second electrode 424B.

### Third Embodiment

Next, a third embodiment of the invention will be described.

In the first embodiment described above, in the receiving transducer 52, the first and second electrodes 422 and 424 are disposed so as to face each other. In contrast, in the third embodiment, an intermediate electrode is disposed between the first and second electrodes 422 and 424. This is a difference from the first embodiment.

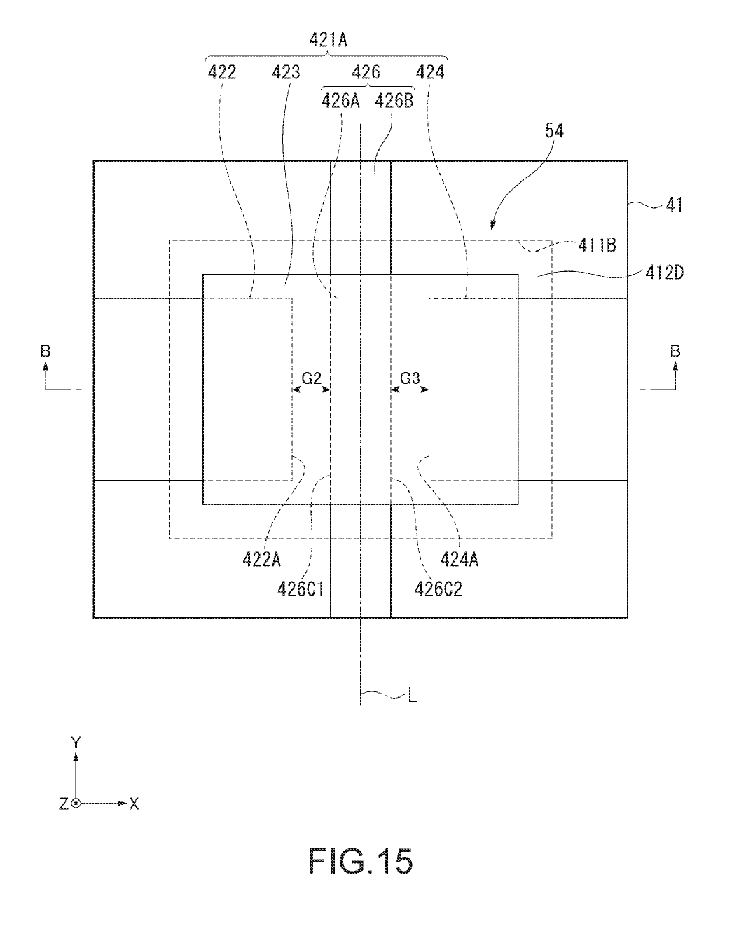

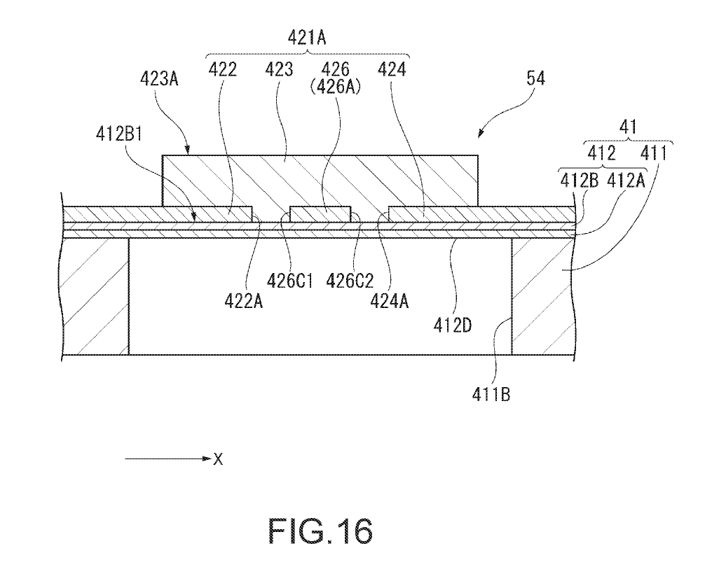

FIG. 15 is a plan view schematically showing a receiving transducer 54 when viewed from the operation surface side of the element substrate 41. FIG. 16 is a schematic sectional view taken along the line B-B in FIG. 15.

As shown in FIGS. 15 and 16, in the receiving transducer 54 of the present embodiment, a receiving element 421A includes a first electrode 422, a second electrode 424, a piezoelectric film 423, and an intermediate electrode 426.

The intermediate electrode 426 is provided on a support film 412 across the inside and outside of an opening 411B along the Y direction in plan view. The intermediate electrode 426 includes an intermediate electrode body portion 426A overlapping the piezoelectric film 423 in plan view and an intermediate lead-out portion 426B extending along the Y direction from the ends of the intermediate electrode body portion 426A on the ±Y side.

The intermediate electrode body portion 426A is disposed at a position, which is equidistant from the electrodes 422 and 424, between the first and second electrodes 422 and 424 in plan view. A −X side end surface 426C1 of the intermediate electrode 426 faces the first end surface 422A of the first electrode 422, and is spaced apart from the first end surface 422A of the first electrode 422 with a gap G2 (second gap) interposed therebetween. In addition, a +X side end surface 426C2 of the intermediate electrode 426 faces the second end surface 424A of the second electrode 424, and is spaced apart from the second end surface 424A of the second electrode 424 with a gap G3 (second gap) interposed therebetween. The sizes (distance between electrodes) of the gaps G2 and G3 are the same.

The intermediate electrode 426 is provided in a range from the receiving region Ar12 to the terminal region Ar2 along the Y direction. That is, the intermediate electrode 426 is a common electrode in a plurality of receiving transducers 54 provided along the Y direction.

In the receiving transducer 54 formed as described above, the first and second electrodes 422 and 424 are connected to a common potential circuit included in the receiving circuit 234 of the wiring substrate 23 through the electrode lines 521 and 522, respectively. Accordingly, the first and second electrodes 422 and 424 are set to have a common potential (for example, 0 potential). That is, the first and second electrodes 422 and 424 function as a common electrode (COM electrode).

On the other hand, the intermediate electrode 426 is connected to the receiving circuit 234 of the wiring substrate 23 in the terminal region Ar2. Accordingly, a signal corresponding to the potential difference between the intermediate electrode 426 and the first electrode 422 and a signal corresponding to the potential difference between the intermediate electrode 426 and the second electrode 424 are detected in the receiving circuit 234 of the wiring substrate 23. That is, the intermediate electrode 426 functions as a signal electrode (SIG electrode) that outputs a signal corresponding to the potential difference described above.

In the present embodiment, an example is shown in which the intermediate electrode 426 is an SIG electrode and the first and second electrodes 422 and 424 are COM:electrodes. However, without being limited thereto, for example, the intermediate electrode 426 maybe used as a COM electrode, and the first and second electrodes 422 and 424 may be made to function as SIG electrodes. In this case, voltage signals output from the first and second electrodes 422 and 424 are added up, and are detected as received signals of ultrasonic waves.

### Effects of the Third Embodiment

In the present embodiment, the intermediate electrode 426 is disposed between the first and second electrodes 422 and 424, and an electrostatic capacitance is formed between the first electrode 422 and the intermediate electrode 426 and between the second electrode 424 and the intermediate electrode 426. In such a configuration, it is possible to increase the areas of facing surfaces between electrodes facing each other. Therefore, it is possible to increase the total electrostatic capacitance of the receiving transducer 52.

Here, assuming that the total electrostatic capacitance of the receiving transducer 52 is C₀ and the stray capacitance in an external circuit (for example, a circuit up to the receiving circuit 234 of the wiring substrate 23) is C₁, the output voltage V detected in the receiving circuit 234 is expressed by the following Equation (6).

$$V = Q / ( {C}_{0} + {C}_{1} ) = ( Q / {C}_{0} ) \times \{ {C}_{0} / ( {C}_{0} + {C}_{1} ) \} \qquad ( 6 )$$

As shown in Equation (6), the output voltage V detected in the receiving circuit 234 is not a value (Q/C₀) that is to be detected originally, but includes an error component based on the stray capacitance C₁.

In contrast, in the present embodiment, since it is possible to increase the total electrostatic capacitance C₀ of the receiving transducer 52 as described above, it is possible to bring the value of C₀/(C₀+₁) in Equation (6) close to “1”. Therefore, since it is possible to suppress the influence of the stray capacitance C₁ of an external circuit, it is possible to avoid a voltage drop in the received signal.

In addition, the gap G2 between the first electrode 422 and the intermediate electrode 426 and the gap G3 between the second electrode 424 and the intermediate electrode 426 are the same. That is, the gaps G2 and G3 are formed such that distances between electrodes in pairs of electrodes forming the electrostatic capacitance are the same. Accordingly, it is possible to suppress a situation in which electric charges concentrate on an electrode pair in which the distance between electrodes is the smallest. Thus, since each electrode pair can be made to function as a capacitor, it is possible to increase the electrostatic capacitance more reliably.

### Fourth Embodiment

Next, a fourth embodiment of the invention will be described.

In the third embodiment described above, one intermediate electrode 426 is disposed between the first and second electrodes 422 and 424. In contrast, in the fourth embodiment, a plurality of intermediate electrodes are disposed between the first and second electrodes. This is a difference from the third embodiment.

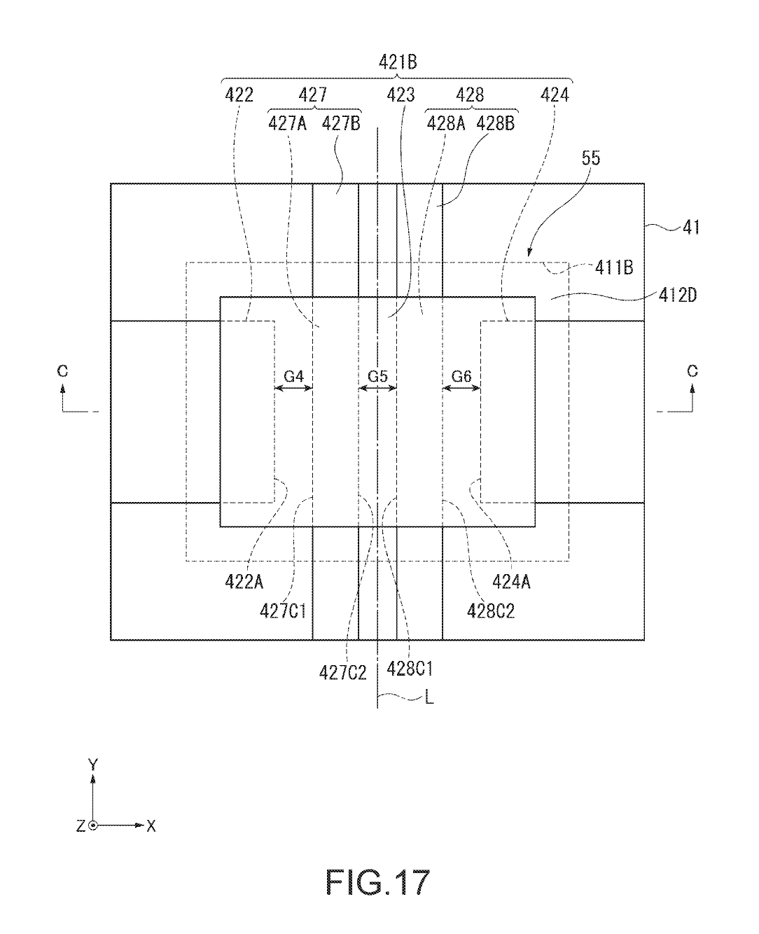

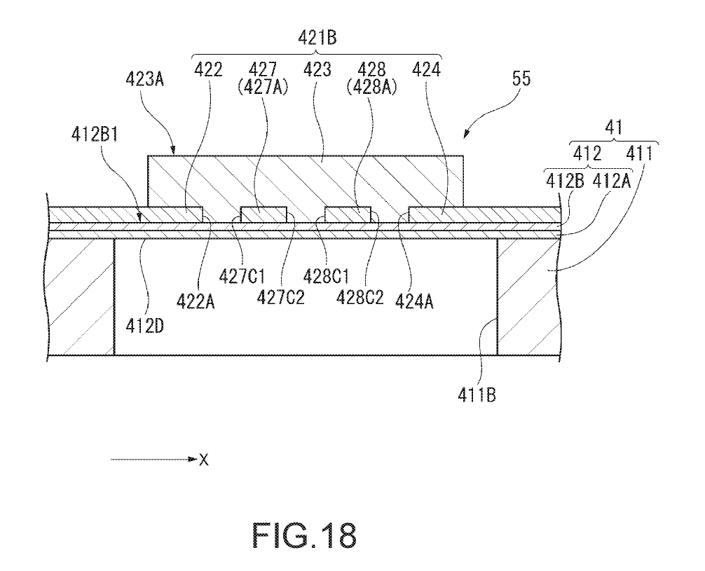

FIG. 17 is a plan view schematically showing a receiving transducer 55 when viewed from the operation surface side of the element substrate 41. FIG. 18 is a schematic sectional view taken along the line C-C in FIG. 17.

As shown in FIG. 17, a receiving element 421B of the receiving transducer 55 of the present embodiment includes not only the first and second electrodes 422 and 424 and the piezoelectric film 423 but also a first intermediate electrode 427 and a second intermediate electrode 428.

The first intermediate electrode 427 is an intermediate electrode according to the invention, and is provided on a support film 412 across the inside and outside of an opening 411B along the Y direction in plan view. The first intermediate electrode 427 is formed in the same manner as the intermediate electrode 426 provided in the receiving transducer 54 of the third embodiment, and includes a first intermediate electrode body portion 427A and a first intermediate lead-out portion 427B. The first intermediate electrode 427 is disposed such that a −X side end surface 427C1 faces the first end surface 422A of the first electrode 422 with a gap G4 (second gap) interposed therebetween.

The second intermediate electrode 428 corresponds to an intermediate electrode according to the invention, and is formed appropriately similar to the first intermediate electrode 427. The second intermediate electrode 428 is disposed on the same plane as the first electrode 422, the second electrode 424, and the first intermediate electrode 427. A −X side end surface 428C1 of the second intermediate electrode 428 is spaced apart from a +X side end surface 427C2 of the first intermediate electrode 427 with a gap G5 (second gap) interposed therebetween in plan view. In addition, a +X side end surface 428C2 of the second intermediate electrode 428 is spaced apart from the second end surface 424A of the second electrode 424 with a gap G6 (second gap) interposed therebetween.

The sizes (distance between electrodes) of the gaps G4, G5, and G6 are the same.

The receiving transducer 55 formed as described above is configured to pass through the center position of the piezoelectric film 423 in plan view along the Y direction and to be axisymmetric with respect to the virtual line L along the Y direction. That is, the first electrode 422, the first intermediate electrode 427, the second intermediate electrode 428, and the second electrode 424 are disposed at equal distances in plan view. In addition, the first intermediate electrode 427 and the second intermediate electrode 428 are disposed so as to interpose the virtual line L therebetween and interpose the center of the piezoelectric film 423 therebetween in plan view. That is, no electrode is formed at a position where the amplitude when the flexible portion 412D vibrates is maximized, which is a position where the amount of distortion of the piezoelectric film 423 is maximized. Therefore, in the present embodiment, it is possible to detect a potential difference, which is generated at the position where the distortion of the piezoelectric film 423 is the largest, with the first intermediate electrode 427 and the second intermediate electrode 428.

In the present embodiment, the first electrode 422 and the second intermediate electrode 428 function as COM electrodes, and are set to have a common potential (for example, 0 potential). On the other hand, the second electrode 424 and the first intermediate electrode 427 function as SIG electrodes, and a signal corresponding to a potential difference between the electrodes is output to the receiving circuit 234 of the wiring substrate 23.

### Effects of the Fourth Embodiment

In the present embodiment, not only the same effects as in the third embodiment but also the following effects are obtained.

That is, in the present embodiment, the intermediate electrodes 427 and 428 are provided at positions not overlapping the center position of the piezoelectric film. 423. That is, since no electrode is disposed at a position where the distortion of the piezoelectric film 423 is maximized, it is possible to increase the output voltage V from the piezoelectric film 423. Accordingly, it is possible to improve detection sensitivity.

### MODIFICATION EXAMPLES

The invention is not limited to the embodiments described above, but various modifications, improvements, and appropriate combinations of the respective embodiments maybe made in a range where the object of the invention can be achieved.

Although the first electrode 422 (422B) and the second electrode 424 (424B) are provided within the same plane in each of the embodiments described above, the invention is not limited to such a configuration.

For example, the first electrode 422 maybe provided on the flexible portion 412D, and the second electrode 424 may be embedded in the piezoelectric film 423.

In the third and fourth embodiments, the configuration is exemplified in which the intermediate electrodes 426, 427, and 428 are provided on the flexible portion 412D for the receiving transducer 52 of the first embodiment. However, the invention is not limited to such a configuration. For example, the intermediate electrodes 426, 427, and 428 may be provided for the receiving transducer 53 of the second embodiment. In this case, it is preferable to provide the intermediate electrodes 426, 427, and 428 on the surface (third surface 425A1) of the first piezoelectric layer 425A facing the second piezoelectric layer 425B. In this case, since it is possible to form simultaneously the first electrode 422B, the second electrode 424B, and the intermediate electrodes 426, 427, and 428, it is possible to suppress the deterioration of the piezoelectric film 425.

In addition, the intermediate electrodes 426, 427, and 428 may be formed on a different plane from the first electrode 422 (422B) or the second electrode 424 (424B) as in the modification example described above.

In the embodiments described above, the configuration is exemplified in which the first end surface 422A of the first electrode 422 (422B) and the second end surface 424A of the second electrode 424 (424B) are parallel. However, the invention is not limited to such a configuration. For example, only parts of the first and second end surfaces 422A and 424A may be parallel.

In the third embodiment described above, the configuration is exemplified in which one intermediate electrode 426 is provided between the first and second electrodes 422 and 424. In addition, in the fourth embodiment described above, the configuration is exemplified in which two intermediate electrodes 427 and 428 are provided between the first and second electrodes 422 and 424. However, three or more intermediate electrodes may be provided.

In this case, however, the second polarization voltage when performing polarization processing on the piezoelectric film 423 (425) is also increased. Therefore, as the number of intermediate electrodes, it is preferable to use one or two intermediate electrodes as in the third or fourth embodiment.

In each of the embodiments described above, PZT that is a perovskite type transition metal oxide is exemplified as a material of the piezoelectric films 423 and 425. However, the material of the piezoelectric films 423 and 425 is not limited thereto.

As a perovskite type transition metal oxide that forms the piezoelectric films 423 and 425, for example, BiBaFeTiO₃, KNaNbO₃, BST (barium strontium titanate: (BaxSr₁₋ₓ) TiO₃ ), and SBT (strontium bismuth tantalate: SrBi₂Ta₂O₉) may be used in addition to the PZT.

In addition, although an example is shown in which the support film 412 is formed by two layers of the support layer 412A and the surface layer 412B. However, the configuration of the support film 412 is not limited thereto.

For example, the support film 412 may be formed by only the surface layer 412B that is a transition metal oxide (ZrO₂), or may be formed by a laminate including three or more layers.

In addition, although an example is shown in which the surface layer 412B is formed as a ZrO₂ layer, the material of the surface layer 412B is not limited thereto. For example, the surface layer 412B may be formed of TiO₂.

In the first embodiment described above, the receiving transducer group 52A is configured such that a plurality of receiving transducers 52 are connected in parallel between the electrode lines 521 and 522. However, the configuration of the receiving transducer group 52A is not limited thereto.

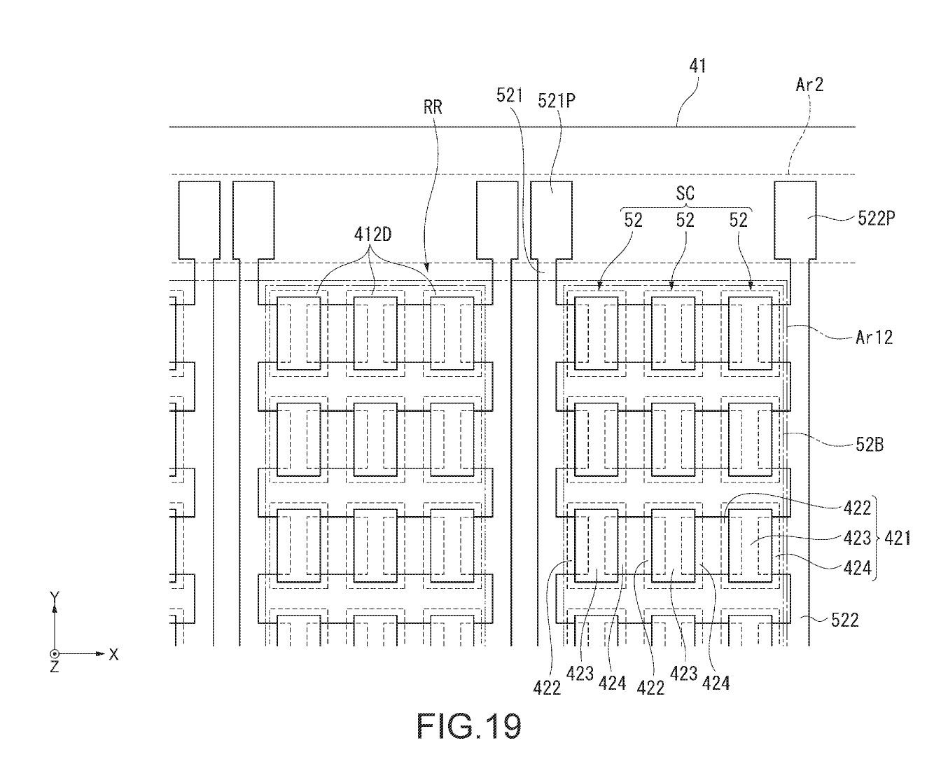

FIG. 19 is a plan view schematically showing a modification example of the receiving array RR.

A receiving transducer group 52B in the example shown in FIG. 19 includes a pair of electrode lines 521 and 522 provided along the Y direction and a series portion SC provided between the pair of electrode lines 521 and 522. In the series portion SC, a plurality of receiving transducers 52 (in the example shown in FIG. 19, three receiving transducers 52) are connected in series along the X direction. A plurality of series portions SC are arranged along the Y direction, and are connected in parallel between a pair of electrode lines 521 and 522.

In such a configuration, since voltage signals output from the respective receiving transducers 52 connected to the series portion SC are added up and the sum voltage is output, it is possible to increase the strength of the received signal. Accordingly, it is possible to improve reception sensitivity.

In the embodiment described above, an example is shown in which the first electrode 422 (422B) and the second electrode 424 (424B) are spaced apart from each other with the gap G1 of 2 μm or more and 8 μm or less interposed therebetween. However, the invention is not limited thereto.

For example, the size of the gap G1 may be less than In this case, however, as described above, since the distance d between electrodes is reduced, the output voltage V of the piezoelectric film 423 (425) maybe reduced. However, it is possible to reduce the second polarization voltage in the polarization processing. In addition, by forming the series portion SC using a plurality of receiving transducers 52 as shown in FIG. 19, it is possible to increase the strength of the received signal.

In addition, in a case where a power supply, which can apply a larger voltage as the second polarization voltage applied to each receiving transducer 52 during the polarization processing, is used as the polarization circuit 235, the size of the gap G1 may be larger than 8 μm.

In each of the embodiments described above, the receiving transducers 52, 53, 53A, 54, and 55 are configured to be approximately axisymmetric with respect to the virtual line L, which is parallel to the Y direction and passes through the center position of the piezoelectric film 423 (425), in plan view. However, the invention is not limited thereto. For example, the center position of the gap G1 between the first electrode 422 (422B) and the second electrode 424 (424B) may be made to overlap the virtual line L in plan view.

In each of the embodiments described above, the receiving transducers 52, 53, 53A, 54, and 55 are configured to include the piezoelectric film 423 (425) having a rectangular shape in plan view and the rectangular first electrode 422 (422B) and the rectangular second electrode 424 (424B). However, the invention is not limited thereto. For example, piezoelectric films having various polygonal shapes, a circular shape, an elliptical shape, and the like in plan view may be used as the piezoelectric body according to the invention. Specifically, a configuration including a piezoelectric film having a circular shape in plan view, a circular first electrode overlapping the center position of the piezoelectric film, and an annular second electrode surrounding at least a part of the periphery of the first electrode may be adopted as a receiving transducer. In addition, an annular intermediate electrode may be provided between the first and second electrodes.

In each of the embodiments described above, an example is shown in which the element substrate 41, the sealing plate 43, the acoustic matching layer 44, and the acoustic lens are common members in the transmission array TR (transmitting transducer 51) and the receiving array RR (receiving transducers 52, 53, 53A, 54, and 55). However, the invention is not limited thereto.

For example, the transmission array TR may be provided on a transmission element substrate, and the receiving array RR may be provided on a receiving element substrate. Similarly, the sealing plate 43, the acoustic matching layer 44, and the acoustic lens 45 may be provided in each of the transmission array TR and the receiving array RR.

In the embodiments described above, the configuration is exemplified in which the acoustic matching layer 44 and the acoustic lens 45 are provided on a side of the support film 412 (flexible portion 412D) not facing the substrate body portion 411. However, the invention is not limited to such a configuration.

For example, the acoustic matching layer 44 and the acoustic lens 45 may be provided on a side of the support film 412 (flexible portion 412D) facing the substrate body portion 411, and the acoustic matching layer 44 may be filled in the openings 411A and 411B. In this case, the sealing plate 43 is provided on a side of the support film 412 not facing the substrate body portion 411, and has grooves at positions facing the openings 411A and 411B in plan view. In such a configuration, each electrode of the transmitting transducer 51 or the receiving transducers 52, 53, 53A, 54, and 55 cannot be exposed to the acoustic matching layer 44 side. Therefore, it is possible to improve waterproofness in the ultrasonic device 22.

In each of the embodiments described above, the ultrasonic measurement apparatus whose measurement target is an organ in the body is exemplified. However, the invention is not limited thereto. For example, the invention can be applied to an ultrasonic measurement apparatus for detecting defects of various structures or for inspecting the aging of various structures with the various structures as measurement targets. In addition, for example, the invention can be applied to an ultrasonic measurement apparatus for detecting defects of a measurement target, such as a semiconductor package or a wafer.

In addition, specific structures when implementing the invention may be formed by appropriately combining the embodiments and the modification examples described above in a range where the object of the invention can be achieved, or may be appropriately changed to other structures in a range where the object of the invention can be achieved.

### EXAMPLES

Evaluation results of reliability for the moisture resistance (dielectric breakdown) according to the invention will be shown below through examples and comparative examples.

### Examples 1 to 3 and Comparative Example 1

In Example 1, the receiving transducer 52 shown in the first embodiment was used.

In Example 2, the receiving transducer 53 shown in the second embodiment was used.

In Example 3, the receiving transducer 53A shown in the modification example of the second embodiment was used.

In Comparative Example 1, a receiving transducer was used in which a piezoelectric film was formed on a flexible portion of a support film and first and second electrodes were disposed on a second surface of the piezoelectric film, which was a surface not facing the flexible portion, so as to face each other.

Here, in each receiving transducer, the conditions of the distance between the first and second electrodes, the conditions of the piezoelectric film, and the conditions of the flexible portion were set to the following conditions.

Distance between the first and second electrodes: 6 μ

Piezoelectric film: PZT having a thickness of 400 nm

Flexible portion: the width of an opening is adjusted such the resonance frequency becomes 8.6 MHz

### Test for Moisture Resistance

Each receiving transducer was placed under the humidity environment of 90% and a moisture resistance test to apply a sine wave voltage, which had amplitude 10 V and a frequency of 1 MHz, between the first and second electrodes was performed to evaluate whether or not dielectric breakdown occurs within one hour. Test results are shown in Table 1. In Table 1, “Bad” indicates a case where dielectric breakdown occurred, “Good” indicates a case where no dielectric breakdown occurred.

TABLE 1

| | Evaluation results |
|---|---|
| Comparative | Bad |
| Example 1 | |
| Example 1 | Good |
| Example 2 | Good |
| Example 3 | Good |

### Evaluation Results

As shown in Table 1, in the Comparative Example 1, breakdown was observed.

Also in the Comparative Example 1, the occurrence of dielectric breakdown can be suppressed by covering a waterproof protective film (for example, Al₂O₃ or Ta₂O₅) with a boundary portion between the piezoelectric film and the electrode. In this case, however, since the piezoelectric film is damaged by the formation of the protective film (crystal defects occur), and the piezoelectric characteristics of the piezoelectric film are degraded.

In contrast, in the Examples 1 to 3, it can be seen that no dielectric breakdown was observed and there was high durability against pulse voltage application under a high humidity. Since the PZT that is a piezoelectric film has water resistance, high waterproofness can be realized by covering the first and second electrodes with the PZT. As a result, the occurrence of dielectric breakdown is suppressed. In addition, since a protective film or the like is not formed on the PZT, the PZT is not damaged in the manufacturing process. Accordingly, it is possible to realize enhanced piezoelectric characteristics.

Next, the relationship between reception sensitivity and the positions of electrodes (first and second electrodes) with respect to the piezoelectric body in the examples and the comparative examples is shown.

### Examples 4 to 8 and Comparative Example 2

In Example 4, the receiving transducer 52 shown in the first embodiment was used.

In Example 5, the receiving transducer 53 shown in the second embodiment was used, and the distance from the support film 412 to electrodes (first and second electrodes 422B and 424B) was set to 80 nm.

In Example 6, the receiving transducer 53 shown in the second embodiment was used, and the distance from the support film 412 to electrodes (first and second electrodes 422B and 424B) was set to 160 nm.

In Example 7, the receiving transducer 53 shown in the second embodiment was used, and the distance from the support film 412 to electrodes (first and second electrodes 422B and 424B) was set to 240 nm.

In Example 8, the receiving transducer 53 shown in the second embodiment was used, and the distance from the support film 412 to electrodes (first and second electrodes 422B and 424B) was set to 320 nm.

In Comparative Example 2, similar to the Comparative Example 1 described above, a receiving transducer was used in which a piezoelectric film was formed on a flexible portion of a support film and first and second electrodes were disposed on a second surface of the piezoelectric film, which was a surface not facing the flexible portion, so as to face each other.

Here, in each receiving transducer, the conditions of the distance between the first and second electrodes, the conditions of the piezoelectric film, and the conditions of the flexible portion were set to the following conditions.

Distance between the first and second electrodes: 6 m

Piezoelectric film: PZT having a thickness of 400 nm

Flexible portion: the width of an opening is adjusted such the resonance frequency becomes 8.6 MHz

### Measurement of Reception Sensitivity

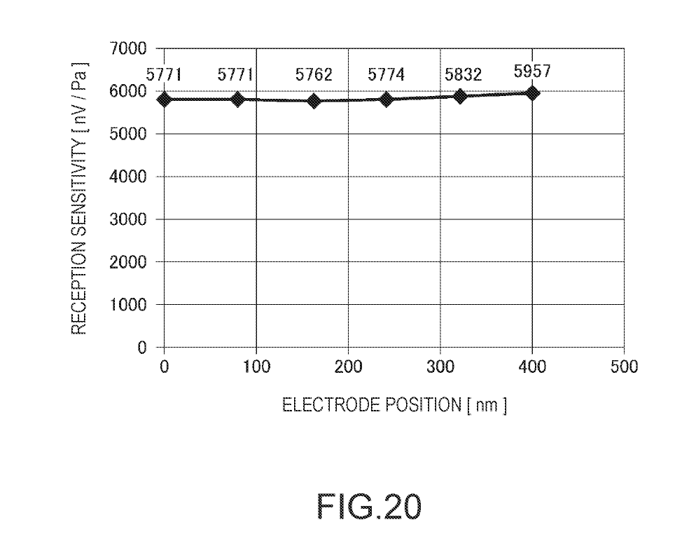

The reception sensitivity of each receiving transducer was calculated using a finite element method (FEM). The calculated reception sensitivity is shown in FIG. 20.

As shown in FIG. 20, it can be seen that the reception sensitivity is not greatly changed even in a case where the distance from the support film 412 to electrodes (first electrodes 422 and 422B and second electrodes 424 and 424B) is changed.

In the related art, it has been considered that the upper surface (surface farthest from the support film 412) of the piezoelectric film where the amount of distortion is the maximum is an optimal electrode formation position. In this case, as described above, there has been a problem of dielectric breakdown or the like due to deterioration of the piezoelectric body during the formation of electrodes or the presence of air between the first and second electrodes. In contrast, the inventors of the invention found that the reception sensitivity of the receiving transducer was approximately constant regardless of the position of each electrode (distance from the support film 412 to the electrode) as shown in FIG. 20. Accordingly, the inventors of the invention solve the problems in the related art by deriving the configuration of the invention. That is, in the invention, it is possible to reduce the degradation of the piezoelectric characteristics or the risk of dielectric breakdown as in each of the above embodiments or Examples 1 to 3 without reducing the reception sensitivity in the invention as shown in FIG. 20. As a result, it is possible to significantly improve the performance and reliability of the receiving transducer.

The entire disclosure of Japanese Patent Application No. 2015-213454 filed Oct. 29, 2015 is expressly incorporated by reference herein.

What is claimed is:

1. A piezoelectric element, comprising:
a flexible film;
a piezoelectric body provided on the flexible film;
a first electrode provided between a first surface of the flexible film, on which the piezoelectric body is provided, and a second surface of the piezoelectric body not facing the flexible film; and
a second electrode that is provided between the first and second surfaces and that faces the first electrode with a first gap interposed therebetween in plan view as viewed from a thickness direction of the flexible film.

2. The piezoelectric element according to claim 1,
wherein the first and second electrodes are provided between the flexible film and the piezoelectric body.

3. The piezoelectric element according to claim 1,
wherein the first and second electrodes are embedded in the piezoelectric element.

4. The piezoelectric element according to claim 1,
wherein the first and second electrodes are provided within a plane parallel to the first surface.

5. The piezoelectric element according to claim 1,
wherein the first electrode has a first end surface facing the second electrode,
the second electrode has a second end surface facing the first electrode, and
the first and second end surfaces are parallel to each other.

6. The piezoelectric element according to claim 1, further comprising:
at least one or more intermediate electrodes that are provided between the first and second electrodes in plan view and that face each of the first and second electrodes with a second gap interposed therebetween in plan view.

7. The piezoelectric element according to claim 1,
wherein the piezoelectric body is formed of a perovskite type transition metal oxide.

8. The piezoelectric element according to claim 7,
wherein the piezoelectric body contains Pb, Zr, and Ti.

9. The piezoelectric element according to claim 1,
wherein the flexible film includes a first layer in contact with the piezoelectric body, and
the first layer is formed of a transition metal oxide.

10. The piezoelectric element according to claim 9,
wherein the first layer is formed of ZrO₂.

11. The piezoelectric element according to claim 1,
wherein the first gap is 2 μm or more and 8 μm or less.

12. A piezoelectric module, comprising:
a flexible film;
a piezoelectric body having a first surface in contact with the flexible film and a second surface opposite to the first surface;
a first electrode provided between the first and second surfaces of the piezoelectric body;
a second electrode that is provided between the first and second surfaces of the piezoelectric body and that faces the first electrode with a first gap interposed therebetween in plan view as viewed from a thickness direction of the flexible film; and
a wiring substrate having a terminal unit to which the first and second electrodes are electrically connected.

13. The piezoelectric module according to claim 12,
wherein the wiring substrate includes a polarization circuit that performs polarization processing by applying an electric field of 10 kV/cm or more between the first and second electrodes.

14. An electronic apparatus, comprising:
a piezoelectric element including a flexible film, a piezoelectric body having a first surface in contact with the flexible film and a second surface opposite to the first surface, a first electrode provided between the first and second surfaces of the piezoelectric body, and a second electrode that is provided between the first and second surfaces of the piezoelectric body and that faces the first electrode with a first gap interposed therebetween in plan view as viewed from a thickness direction of the flexible film; and
a control unit that controls the piezoelectric element.

15. A piezoelectric element manufacturing method, comprising:
forming, on a flexible film, a first electrode and a second electrode, which faces the first electrode with a first gap interposed therebetween in plan view as viewed from a thickness direction of the flexible film; and
forming a piezoelectric body, which covers a part of the first electrode and a part of the second electrode, on the flexible film.

16. A piezoelectric element manufacturing method, comprising:
forming a first piezoelectric layer on a flexible film;
forming a first electrode and a second electrode, which faces the first electrode with a first gap interposed therebetween in plan view as viewed from a thickness direction of the flexible film, on the first piezoelectric layer; and
forming a second piezoelectric layer, which covers a part of the first electrode and a part of the second electrode, on the first piezoelectric layer.
